(19) United States
(12) Patent Application Publication
(10) Pub. No.: US 2024/0271212 A1
(43) Pub. Date: Aug. 15, 2024

(54) CHARACTERIZATION AND TREATMENT OF ASTHMA

(71) Applicant: The University of Chicago, Chicago (IL)

(72) Inventors: Carole OBER, Chicago, IL (US); Selene CLAY, Chicago, IL (US); Nathan SCHOETTLER, Chicago, IL (US)

(21) Appl. No.: 18/565,970

(22) PCT Filed: Jun. 14, 2022
(86) PCT No.: PCT/US2022/033442

§ 371 Date: Nov. 30, 2023

Related U.S. Application Data
(60) Provisional application No. 63/210,343, filed on Jun. 14, 2021.

(51) Int. Cl. C12Q 1/6883; A61K 31/7105

(52) U.S. Cl. CPC C12Q 1/6883; A61K 31/7105; C12Q 2600/156; C12Q 2600/158

(57) ABSTRACT

Provided herein are compositions and methods for the characterization and treatment of asthma.

### CROSS-REFERENCE TO RELATED APPLICATIONS

This application claims the benefit of U.S. Provisional Patent Application No. 63/210,343, filed on Jun. 14, 2021, which is incorporated by reference herein.

### STATEMENT REGARDING FEDERAL FUNDING

This invention was made with government support under AI114271, OD023282, and HL085197 awarded by the National Institutes of Health. The government has certain rights in the invention.

### FIELD

Provided herein are compositions and methods for the characterization and treatment of asthma.

### BACKGROUND

Asthma is a chronic, inflammatory disease of the airways, affecting over 330 million people worldwide and representing a significant global health burden (Ref. 1: incorporated by reference in its entirety). Genome-wide association studies (GWASs) have reported over 150 independent loci associated with asthma (Refs. 2-6; incorporated by reference in their entireties), including strong and replicated associations at the human leukocyte antigen (HLA) region on chromosome 6p21. Recently, Pividori et al. (Ref. 3: incorporated by reference in its entirety) performed a GWAS for childhood-onset asthma (COA) and adult-onset asthma (AOA) in individuals from the UK Biobank (UKB) (Ref. 7: incorporated by reference in its entirety) and reported independent associations at the HLA class I (HLA-C/B) and class II (HLA-DR/DQ) regions in both. Even though variants in the class II region were more significantly associated and had greater effect sizes with COA than with AOA, the HLA class II region was the most significant locus for AOA (ref. 3: incorporated by reference in its entirety). In contrast, associations with variants in the class I region were more similar in COA and AOA. Despite robust associations in asthma GWASs with variation in the HLA region, the specific variants and genes contributing to asthma risk are not known to the field.

The HLA region is the most frequently associated locus with asthma and allergic diseases (Ref. 8: incorporated by reference in its entirety). Whereas its central role in adaptive immunity has been extensively characterized (Refs. 9-11: incorporated by reference in their entireties), determining the causal variants and their putative functions has been particularly challenging due to the remarkably high gene density, extraordinary levels of polymorphism, and striking linkage disequilibrium (LD) that characterize this region (Refs. 12-13; incorporated by reference in their entireties). These features make it especially difficult to determine which disease-associated variants are causal and which genes underlie associations. Additionally, GWASs typically focus on associations with individual single nucleotide polymorphisms (SNPs), which do not fully capture the complexity of allelic variation at HLA genes.

### SUMMARY

Provided herein are compositions and methods for the characterization and treatment of asthma.

In some embodiments, provided herein are methods of treating or preventing adult-onset asthma (AOA) or another disease or condition comprising modulating (e.g., inhibiting, increasing) the activity and/or the expression of human leukocyte antigen (HLA) class II histocompatibility antigen, DQ alpha 2 chain (HLA-DQA2) and/or HLA class II histocompatibility antigen, DQ beta 2 chain (HLA-DQB2). In some embodiments, the activity and/or the expression of HLA-DQA2 and/or HLA-DQB2 is inhibited in lung epithelial cells, lymphoblastoid cells, peripheral blood mononuclear cells (PBMCs), upper airway (nasal) epithelial cells (NECs), lower airway (bronchial) epithelial cells (BECs), Langerhans cells, etc.

Many embodiments herein provide for inhibiting the activity and/or expression of HLA-DQA2 and/or HLA-DQB2, for example, for the treatment of asthma: however, in certain embodiments herein, it will be understood that the activity and/or expression of HLA-DQA2 and/or HLA-DQB2 are enhanced for the treatment and/or prevention of other immune-mediated conditions. In some embodiments, the compositions and methods described herein for the inhibition of HLA-DQA2 and/or HLA-DQB2 may also find use in the enhancement of HLA-DQA2 and/or HLA-DQB2 activity and/or expression.

In some embodiments, inhibiting the activity of HLA-DQA2 and/or HLA-DQB2 comprises inhibiting the binding of one or more antigenic peptides present in HLA-DQA2 and/or HLA-DQB2 from binding and/or being recognized by immune cells. In some embodiments, inhibiting the activity of HLA-DQA2 and/or HLA-DQB2 comprises administering a HLA-DQA2 and/or HLA-DQB2 inhibitor. In some embodiments, the HLA-DQA2 and/or HLA-DQB2 inhibitor is a small molecule, peptide, or antibody (or antibody fragment). In some embodiments, provided herein are HLA-DQA2 and/or HLA-DQB2 inhibitors for use in the methods described herein.

In some embodiments, inhibiting the expression of HLA-DQA2 and/or HLA-DQB2 comprises targeting the HLA-DQA2 and/or HLA-DQB2 genes and reducing expression thereof. In some embodiments, reducing expression of HLA-DQA2 and/or HLA-DQB2 comprises administering a nucleic acid inhibitor of gene expression. In some embodiments, provided herein are nucleic acid inhibitors of HLA-DQA2 and/or HLA-DQB2 gene expression. In some embodiments, targeting the HLA-DQA2 and/or HLA-DQB2 genes comprises knocking down expression by RNAi or CRISPR. In some embodiments, inhibiting the expression of HLA-DQA2 and/or HLA-DQB2 comprises targeting one or more SNPs that are causal variants of the increased expression of HLA-DQA2 and/or HLA-DQB2. In some embodiments, the one or more SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580), rs17612625, rs17612633, rs17612781, rs17612788, rs17612802, rs17612858, rs9273326, rs9273329, rs9273330, rs9273339, rs3828789, and rs9274660. In some embodiments, targeting the one or more SNPs comprises editing one or more of the genes containing the SNPs. In some embodiments, one or more of the genes containing the SNPs is edited by CRISPR. In some embodiments, provided herein are gene editing agents for use in the method the methods herein.

In some embodiments, the methods herein find use in the treatment of prevention of a disease or condition, such as asthma (e.g., COA, AOA, etc.), allergies (e.g., seasonal allergies, food allergies, etc.), cancer, autoimmune disease (e.g., celiac, rheumatoid arthritis, etc.).

In some embodiments, provided herein are methods comprising: (a) obtaining a sample from a subject: (b) detecting the level of HLA-DQA2 and/or HLA-DQB2 gene expression in the sample; and (c) comparing the level of HLA-DQA2 and/or HLA-DQB2 gene expression in the sample to a control or threshold level. In some embodiments, methods further comprise (d) assessing the risk of the subject developing adult-onset asthma (AOA) based on the comparison of step (c), wherein if the level of HLA-DQA2 and/or HLA-DQB2 gene expression in the sample is greater than a control or threshold level then the subject is at increased risk of developing AOA. In some embodiments, methods further comprise (d) characterizing the type of asthma the subject suffers from based on the comparison of step (c), wherein if the level of HLA-DQA2 and/or HLA-DQB2 gene expression in the sample is greater than a control or threshold level then the subject suffers from a HLA-DQA2- and/or HLA-DQB2-dependent AOA. In some embodiments, methods further comprise (d) determining a treatment for the subject based on the comparison of step (c), wherein an increased level of HLA-DQA2 and/or HLA-DQB2 gene expression in the sample compared to the control or threshold level indicates treatment by the compositions or methods herein. In some embodiments, the level of HLA-DQA2 and/or HLA-DQB2 gene expression is measured by quantitative PCR (qPCR).

In some embodiments, provided herein are methods comprising: (a) obtaining a sample from a subject: (b) detecting the level of HLA-DQA2 and/or HLA-DQB2 protein in the sample; and (c) comparing the level of HLA-DQA2 and/or HLA-DQB2 protein in the sample to a control or threshold level. In some embodiments, methods further comprise (d) assessing the risk of the subject developing adult-onset asthma (AOA) based on the comparison of step (c), wherein if the level of HLA-DQA2 and/or HLA-DQB2 protein in the sample is greater than a control or threshold level then the subject is at increased risk of developing AOA. In some embodiments, methods further comprise (d) characterizing the type of asthma the subject suffers from based on the comparison of step (c), wherein if the level of HLA-DQA2 and/or HLA-DQB2 protein in the sample is greater than a control or threshold level then the subject suffers from a HLA-DQA2- and/or HLA-DQB2-dependent AOA. In some embodiments, methods further comprise (d) determining a treatment for the subject based on the comparison of step (c), wherein an increased level of HLA-DQA2 and/or HLA-DQB2 protein in the sample compared to the control or threshold level indicates treatment by the compositions or methods herein. In some embodiments, the level of HLA-DQA2 and/or HLA-DQB2 protein is measured by any suitable method, such as, spectrometry methods (e.g., HPLC, mass spectrometry, etc.), antibody-dependent methods (e.g., ELISA, protein immunoprecipitation, Western blot, protein immunostaining, etc.), etc.

In some embodiments, provided herein are methods comprising: (a) obtaining a sample from a subject; and (b) detecting in the sample the presence/absence of one or more SNPs that are causal variants of the increased expression of HLA-DQA2 and/or HLA-DQB2. In some embodiments, methods further comprise (c) assessing the risk of the subject developing adult-onset asthma (AOA) based on the presence/absence of one or more SNPs that are causal variants of the increased expression of HLA-DQA2 and/or HLA-DQB2, wherein the presence of one or more of the causal variants in the sample is indicative of an increased risk of developing AOA. In some embodiments, methods further comprise (c) characterizing the type of asthma the subject suffers from based on the presence/absence of one or more SNPs that are causal variants of the increased expression of HLA-DQA2 and/or HLA-DQB2, wherein the presence of one or more of the causal variants in the sample indicates that the subject suffers from a HLA-DQA2- and/or HLA-DQB2-dependent AOA. In some embodiments, methods further comprise (c) determining a treatment for the subject based on the presence/absence of one or more SNPs that are causal variants of the increased expression of HLA-DQA2 and/or HLA-DQB2, wherein the presence of one or more of the causal variants in the sample indicates treatment by compositions or methods described herein. In some embodiments, the one or more SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and rs9274660.

In some embodiments, provided herein are methods of assessing the risk of a subject developing a disease or condition (e.g., asthma (e.g., AOA, COA, etc.), an autoimmune disease, allergies, cancer, etc.) comprising: (a) testing a sample from the subject for biomarkers described herein (e.g., presence/absence of SNPs, gene expression, etc.); and (b) assessing the subject's risk. In some embodiments, the biomarkers are selected from (1) one or more SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580), rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and rs9274660; (2) expression level of HLA-DQA2: (3) expression level of HLA-DQB2: (4) presence/absence of HLA-DQA1*0301: (5) the presence/absence of alanine (risk) or serine (protection) at position 31 of HLA-C: etc. In some embodiments, any combination of the biomarkers are analyzed. In some embodiments, assessing the subject's risk comprises: (i) calculating a risk score based on the biomarkers analyzed; and (ii) comparing the risk score to a threshold to determine the subject's risk. In some embodiments, the presence of any biomarker is weighted according to an odds ratio in order to calculate the risk score.

In some embodiments, provided herein are methods of preventing a subject from developing asthma comprising: (1) assessing the risk of developing asthma (e.g., AOA, COA, etc.) by the method of described herein; and (2) administering a prophylactic regime to reduce the subject's risk for developing asthma. In some embodiments, the prophylactic regime comprises one or more of: cessation of tobacco smoking, avoidance of allergens, immunotherapy allergy shots (e.g., reslizumab (CINQAIR), mepolizumab (NUCALA), omalizumab (XOLAIR), etc.), taking asthma medications (e.g., bronchodilators, anticholinergics, leukotriene modifiers, mast cell stabilizers, theophylline, etc.), etc.

Embodiments of the present disclosure include a method of predicting asthma risk in a subject. In accordance with these embodiments, the methods include detecting the presence and/or quantifying levels of one or more biomarkers in a sample from a subject: calculating a risk score based on analysis of the one or more biomarkers; and determining subject's asthma risk (e.g., risk of developing AOA, COA, etc.). In some embodiments, the subject is assigned a risk level, such as low risk, intermediate risk, or high risk based on the calculated risk score. In some embodiments, the biomarkers are weighted in the risk score calculation.

Embodiments of the present disclosure also include a biomarker panel for determining asthma risk in a subject. In accordance with these embodiments, the panel includes at least two of the following biomarkers: (1) one or more SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and rs9274660; (2) expression level of HLA-DQA2: (3) expression level of HLA-DQB2: (4) presence/absence of HLA-DQA1*0301: (5) the presence/absence of alanine (risk) or serine (protection) at position 31 of HLA-C: etc.

In some embodiments, the present disclosure provides a risk score, based on the presence/absence of one or more biomarkers (e.g., SNPs) and/or the level of expression of one or ore biomarkers to determine a subject's risk (e.g., low, intermediate, high, etc.) of developing asthma, thereby permitting selection of appropriate therapies to treat the subject.

### BRIEF DESCRIPTION OF THE DRAWINGS

FIG. 1A-C. HLA Class I Fine-Mapping Results for Childhood- and Adult-Onset Asthma. In a) Childhood-onset asthma and b) Adult-onset asthma, the upper panels show the −log 10(p-values) from the GWAS for 9,021 variants (SNPs, HLA alleles, amino acid polymorphisms) in the HLA class I region: the lower panel shows the fine-mapping posterior inclusion probabilities (PIPs) for the same variants. The dashed line is at genome-wide significance. The colors represent the level-95% credible sets (CS1=red, CS2=blue). c) The PIP, p-value, OR, 95% CI, Allele (non-risk/risk), and risk allele frequency (RAF) are shown for each variant in each CS.

FIG. 2A-C. HLA Class II Fine-Mapping Results for Childhood- and Adult-Onset Asthma. The −log 10(p-values) and PIPs for 10,428 variants in the HLA Class II region are shown for a) Childhood-onset asthma and b) Adult-onset asthma. The colors represent the different credible sets (CS1=orange, CS2=cyan for COA: CS1=magenta, CS2-green for AOA). c) Only the SNPs with the highest PIP in the adult-onset CSs are shown.

FIG. 3A-C. eQTL Fine-Mapping. Upper panels show eQTL −log 10(p-value) and PIPs for each variant (y-axes) tested within +0.5 Mb of the gene transcription start site (TSS). The colored outlines show the eQTL CSs: the magenta points are SNPs that were also in the class II AOA CS1. A purple outline around a magenta point shows the variants shared between the eQTL CS and the class II AOA CS1. The green and blue outlines indicate other CSs. X-axis position in Mb (hg19). The Venn diagrams show the number of variants in the eQTL CS and the number that overlaps with the SNPs in the class II AOA CS1. Results are shown for a) HLA-DQB2 and b) HLA-DQA2. c) Normalized expression of HLA-DQA2 (upper panel) and HLA-DQB2 (lower panel) in LCLs by the number of asthma-risk alleles for rs9272346, a representative class II AOA CS1 SNP.

FIG. 4. Overlaps of Class II AOA CS1 SNPs, eQTL CS SNPs, and functional annotations. The four SNPs in the class II AOA CS1 and in at least one eQTL CS that overlapped with functional annotations are shown at the top of the figure. The colors above each SNP (rsID) indicate which eQTL CS they were in. All four SNPs overlapped with a strong enhancer in GM12878 cells (LCLs: bottom track). LCL H3K4me1, H3K4me3, and H3K27ac y-axis are 0-100. The GM12878 ChromHMM results correspond to: red, active promoter: light red, weak promoter: orange, strong enhancer: yellow, weak/poised enhancer; dark green, transcriptional transition/elongation: light green: weak transcribed. Chromosome position in hg19.

FIG. 5A-C. Localization of Asthma-Associated Amino Acid Variants. Ribbon-figure representations of the peptide-binding pocket are shown for each HLA protein, and the amino-acid variant in focus is highlighted. a) HLA-C p.11, shown in blue, is located within the peptide-binding pocket of the HLA-C molecule (forest green). b) HLA-DQB1 p.55, shown in magenta, lies in the region that may interact with the T-cell receptor on the HLA-DQB1 protein (grey) in complex with HLA-DQA1 (blue). c) HLA-DQA1 p.26, p.47, p.56, and p.76 (green) shown on the HLA-DQA1 protein (blue). p.26 lies in the peptide-binding pocket, p. 76 in the region that may interact with the TCR, and p.56 and p.47 in regions outside of the peptide-binding pocket.

FIG. 6. Fine-Mapping Simulations in the HLA Region. Each point is a variant examined in the class I or class II region using covariate effects estimated from a logistic regression for either childhood-onset asthma (COA) and adult-onset asthma (AOA). The colors represent the credible sets (CS) detected by SuSiE, with the designated effect variant(s) in red, showing the PIPs for each variant. Three randomly selected variants were selected across the class I and class II regions for COA and AOA. SuSiE correctly identified all 3 CSs in each of the 4 simulations, with the causal variant in each CS having the highest PIP in 10 of 12 CSs.

FIG. 7. Expression of HLA-DQB2 and HLA-DQA2. Normalized expression of each gene by the number of asthma-risk alleles for rs9272346 (for LCLs, PBMCs) and rs9274660 (NECs), which were representative class II AOA CS1 SNPs.

FIG. 8. eQTL Fine-Mapping Results with COA and AOA CS SNPs. Upper panels show −log 10(p-value) for each variant and bottom panel shows PIPs for each variant tested within +0.5 Mb of the gene transcription start site. The colored outlines show the eQTL CSs, and a purple outline around a magenta point shows the variants shared between the eQTL CS and class II AOA CS1. SNPs in the class II COA and AOA CSs are shown in different colors (see legend). X-axis position in Mb (hg19).

FIG. 9A-F. ENCODE ChromHMM Results for SNPs in Childhood-Onset and Adult-Onset Credible Sets Vertical red line indicates the location of each SNP. Layered H3K4Me1, H3K4ME3, and H3K27Ac marks and ChromHMM states are shown for the GM12878 cells. Red: active promoter, light red: weak promoter, orange: strong enhancer, yellow: weak/poised enhancer, blue: insulator, dark green: transcriptional transition/elongation, light green: weak transcribed, gray: polycomb-repressed, light gray: heterochromatin/low signal. Asterisk denotes rsID with the highest PIP. a) rs2428494 (shared class I CS) was predicted to reside in a weak promoter, b) rs28481932 (class I COA CS2) in a weakly transcribed region, and c) rs28407950 (class II COA CS1) in an strong enhancer. d) Class II childhood-onset CS2 SNPs were predicted to reside in polycomb-repressed, active promoter, polycomb-repressed, insulator, and weakly transcribed regions (from left to right). e) Class II adult-onset CS1 SNPs. The red or orange mark next to the rsID indicates it is predicted to reside in an active promoter or strong enhancer, respectively. Magenta\hat{ } indicates if it was an eQTL in our study. f) Class II adult-onset CS2 SNPs.

FIG. 10A-B. Class II conditional analyses for (A) adult-onset asthma (AOA) and (B) childhood-onset asthma (COA). Results show the −log₁₀(p-value) for each variant after conditioning on rs9272346 (representative AOA CS1 SNP), and/or HLA-DQA1*03:01 (representative AOA CS2 variant). Colored outlines correspond to different credible sets; AOA CS1: magenta, AOA CS2: green, COA CS1: orange, and COA CS2: cyan. See FIG. 1 for comparison.

FIG. 11. Single cell RNA sequencing (scRNAseq) analysis identified HLA-DQA2 and HLA-DQB2 expression in a subset of lung immune cells. Lung immune cells isolated from two organ donors (602 and 616) whose lungs were not used for transplantation were cultured in media alone (Untreated), lipopolysaccharide (LPS) for 4 hours or LPS for 18 hours. Cells were multiplexed using 10× Genomics CellPlex, pooled, processed using the 10× Genomics platform targeting 12,000 cells and 3′ RNA sequencing libraries generated. Libraries were sequenced using a NovoSeq 6000 sequencer (Illumina) and reads aligned using CellRanger 6.1.1 software (10× Genomics). Data is projected using uniform manifold approximation and projection- (UMAP-) 1 on the x-axis and UMAP-2 on the y-axis. Each point is an individual cell. Upper left, graph-based clustering of cells into 14 distinct clusters. Upper right, cells colored according to the sample and treatment-timepoint identity. Bottom left, cells colored according to the log₂ expression level of HLA-DQA2. Bottom right, cells colored according to the log₂ expression level of HLA-DQB2. Although low levels of expression of HLA-DQA2 and/or HLA-DQB2 were observed in macrophages across many clusters of cells, the highest expression levels of these genes and the highest proportion of cells expressing these genes was in Cluster 13.

FIG. 12. Cells in Cluster 13 had high levels of expression of the classical MHC class 2 genes HLA-DRA (top left), HLA-DRB1 (top right), HLA-DQA1 (middle left), HLA-DQB1 (middle right), HLA-DPA1 (bottom left) and HLA-DPB1 (bottom right). High expression of these genes is a feature of antigen presenting dendritic cells and dendritic-like cells: enrichment of HLA-DQA2 and/or HLA-DQB2 expressing cells in Cluster 13 indicates that these genes are expressed in antigen presenting cells.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a biomarker” is a reference to one or more biomarkers and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

As used herein, the term “preventing” refers to prophylactic steps taken to reduce the likelihood of a subject (e.g., an at-risk subject) from developing or suffering from a particular disease, disorder, or condition (e.g., asthma). The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating.” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

The terms “biological sample,” “sample,” and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), mucosal biopsy tissue and brushed cells, sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the foregoing. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed: exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. It will be appreciated that obtaining a biological sample from a subject may comprise extracting the biological sample directly from the subject or receiving the biological sample from a third party.

As used herein, the term “biomarker” refers to a measurable analyte, the detection of which indicates a particular disease/condition or risk of developing/having a particular disease/condition. A “biomarker” may indicate a change in expression level or state of the measurable substance that correlates with the prognosis of a disease. A “biomarker” may be a protein or peptide, a nucleic acid, or a small molecule. A “biomarker” may be measured in a bodily fluid such as plasma, and/or in a tissue (e.g., mammary tissue). In the context of the method described herein, a “biomarker” can be a SNP, the presence/absence of which is detected in a sample form a subject. In other embodiments herein, a “biomarker” is a gene or protein, the level of expression of which is monitored/quantified. In some embodiments, a biomarker is a genetic variant such as a SNP, insertion, deletion, or repeat.

As used herein, the term “SNP” or “single nucleotide polymorphism” refers to a genetic variation between individuals: e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc. A “polymorphism” is a locus that is variable: that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele inversely correlates with a trait when it is linked to it and when presence of the allele is an indicator that a trait or trait form will not occur in an individual comprising the allele.

A marker polymorphism or allele is “correlated” or “associated” with a specified phenotype (e.g., increased likelihood of developing asthma (e.g., AOA, COA, etc.), increased expression of HLA-DQA2 and/or HLA-DQB2, etc.) when it can be statistically linked (positively or inversely) to the phenotype. That is, the specified polymorphism occurs more commonly in a case population than in a control population. This correlation is often inferred as being causal in nature, but it need not be—simple genetic linkage to (association with) a locus for a trait that underlies the phenotype is sufficient for correlation/association to occur.

As used herein, the term “causal variant” refers to a genetic variation between individuals that result in phenotypic differences.

As used herein, the term “polygenic risk score” refers to a calculated value that is used to define an individuals' risk of developing a disease or condition, based on a multiple biomarkers, each of which might have modest individual effect sizes contribute to the disease or condition, but in aggregate have significant predicting value.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be by parenteral administration (e.g., intravenously, subcutaneously, etc.), orally, etc.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “instructions for administering,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

As used herein, the term “antibody” refers to a whole antibody molecule or a fragment thereof (e.g., fragments such as Fab, Fab′, and F(ab′)2), it may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.): Chothia, C., and Lesk, A. M. (1987) J. Mol. Biol. 196:901-917: or Chothia, C. et al. Nature 342:878-883 (1989). In the present application, the term “CDR” refers to a CDR from either the light or heavy chain, unless otherwise specified.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256: 495-499: herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597: herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.): herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the CHI and variable region of one heavy chain. The heavy chain of a Fab molecule cannot form a disulfide bond with another heavy chain molecule. A “Fab” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203: herein incorporated by reference in their entireties. In certain instances, a single variable region (e.g., a heavy chain variable region or a light chain variable region) may have the ability to recognize and bind antigen.

Other antibody fragments will be understood by skilled artisans.

### DETAILED DESCRIPTION

Provided herein are compositions and methods for the characterization and treatment of asthma.

Genome-wide association studies of asthma have described robust associations with variation across the human leukocyte antigen (HLA) complex. However, specific variants and genes contributing to risk are unknown. Genetic fine mapping analyses were conducted during development of embodiments herein for childhood-onset asthma (COA) and adult-onset asthma (AOA) in individuals from the UK Biobank and expression quantitative trait locus fine-mapping was performed in immune and airway cells. The studies revealed both shared and distinct causal variation between COA and AOA in the HLA class I region and distinct causal variation in the class II region, indicating that expression levels and amino acid variation contribute to risk in both HLA regions and highlighting an important role for the nonclassical class II HLA-DQA2/DQB2 genes in AOA.

Experiments conducted during development of embodiments herein demonstrate that increased expression of HLA-DQA2 and/or HLA-DQB2 is causative of AOA and identified a number of SNPs, the presence of which correlate with the increased expression of HLA-DQA2 and/or HLA-DQB2 and increased risk of AOA. Provided herein are methods of characterizing AOA and/or determining if a subject is at increased risk of developing AOA by identifying the presence/absence of one or more of the SNPs identified herein and/or detecting increased expression of HLA-DQA2 and/or HLA-DQB2 in a sample form a subject. Also provided herein are methods of treating or preventing AOA by correcting the causal variant SNPs, inhibiting the expression of HLA-DQA2 and/or HLA-DQB2, and/or inhibiting the activity of HLA-DQA2 and/or HLA-DQB2. In some embodiments, the methods of treating or preventing AOA herein are combined with known methods of treating, preventing, and/or monitoring for asthma.

Experiments conducted during development of embodiments herein demonstrate that presence of the risk allele at position 31 of HLA-C (alanine instead of serine) is causative of COA. Provided herein are methods of characterizing COA and/or determining if a subject is at increased risk of developing COA by identifying the presence/absence of the HLA-C risk allele in a sample form a subject. Also provided herein are methods of treating or preventing COA by correcting the risk allele, inhibiting the expression of HLA-C, and/or inhibiting the activity of HLA-C in subjects expressing the risk allele. In some embodiments, the methods of treating or preventing COA herein are combined with known methods of treating, preventing, and/or monitoring for asthma.

Experiments conducted during development of embodiments herein demonstrate a panel of SNPs that correlate with both (1) increased expression of HLA-DQA2 and/or HLA-DQB2 and (2) increased likelihood of developing AOA. In some embodiments, a panel comprises one or more (e.g., 1, 2, 3, 4, 5, 6, 7, 8, 9, 10, 15, 20, 25, 30, 35, 40, 45, 50, or more, or ranges therebetween) SNPs selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and/or rs9274660). In some embodiments, a panel comprises at least 4, 8, 12, 16, 20, 30, 40, 50, 100, or more SNPs. In some embodiments, a panel comprises fewer than 500, 200, 100, 50, 20, or 10 SNPs. In some embodiments, Table 4 provides specific cell types in which the SNPs were identified. In some embodiments, methods herein comprise detecting one or more SNPs in the same or related cell types to those there were identified in.

In some embodiments, the disclosure provides a method comprising: (a) obtaining a biological sample from a subject; and (b) assaying the sample for one or more SNP biomarkers described herein (e.g., SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and/or rs9274660). As disclosed herein, the biological sample may be any biological material obtained or otherwise derived from an organism (e.g., a human). The biological sample may comprise, for example, saliva, blood, or a processed blood product. In some embodiments, the sample comprises epithelial cells, such as lung epithelial cells. In some embodiments, the sample comprises lymphoblastoid cells, peripheral blood mononuclear cells (PBMCs), upper airway (nasal) epithelial cells (NECs), lower airway (bronchial) epithelial cells (BECs), Langerhans cells, etc. In some embodiments, obtaining a biological sample from a subject comprises extracting the biological sample directly from the subject or receiving the biological sample from a third party. In other embodiments, a biological sample may be extracted directly from a subject and sent to a third party for analysis.

In some embodiments, methods herein comprise detecting one or more of the SNP biomarkers disclosed herein in an appropriate sample from a subject. In some embodiments, methods herein comprise calculating a risk score (e.g., risk of developing asthma (e.g., AOA, COA) or another disease or condition, etc.) based on the presence/absence of a combination of the biomarkers herein. In some embodiments, biomarkers contribution to the risk score is weighted by a factor related to the degree of correlation to a particular condition (e.g., asthma (e.g., AOA, COA), etc.). In some embodiments, the biomarkers are weighted according to their effect estimate, odds ratio, or any other suitable measure of correlation. In some embodiments, a polygenic risk score is calculated.

Exemplary methods for detecting the presence or absence of a biomarker include, but are not limited to, polymerase chain reaction (PCR)-based technologies including, for example, reverse transcription PCR (RT-PCR) and quantitative or real-time RT-PCR (RT-qPCR). Other methods include microarray analysis, RNA sequencing (e.g., next-generation sequencing (NGS)), in situ hybridization, and Northern blot. In some embodiments, nucleic acid (e.g., DNA or RNA) may be isolated, purified, and/or amplified from the biological sample prior to assaying the biological sample. Commercially available kits and systems for isolating and purifying nucleic acid (e.g., DNA or RNA) may be used in connection with the disclosure.

In some embodiments, primers, probes, or other reagents for detecting the biomarkers herein are provided. The polymorphisms, corresponding marker probes, amplicons or primers described herein can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods herein, the set of marker probes or primers optionally detects a plurality of polymorphisms. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

In some embodiments, a risk score is compared to a threshold level and the subject is diagnosed as being at elevated risk or reduced risk of a condition based thereon (e.g., elevated risk of developing AOA or COA, etc.). The terms “threshold level” and “reference level” may be used interchangeably herein to refer to an assay value that is used to assess diagnostic, prognostic, or therapeutic efficacy and that has been linked or is associated herein with various clinical parameters. It is well-known that threshold levels may vary depending on the nature of the assay and that assays can be compared and standardized.

Some embodiments herein involve detection and analysis of multiple genetic variants (e.g., SNPs). In some embodiments, the presence/absence of multiple SNPs are used to calculate a polygenic risk score suitable for identifying individuals at a greater or lesser risk of developing a condition (e.g., COA. AOA, etc.). Detection methods for detecting relevant alleles include a variety of methods well known in the art, e.g., gene amplification technologies. For example, detection can include amplifying the polymorphism or a sequence associated therewith and detecting the resulting amplicon. This can include admixing an amplification primer or amplification primer pair with a nucleic acid template isolated from the organism or biological sample (e.g., comprising the SNP or other polymorphism), where the primer or primer pair is complementary or partially complementary to at least a portion of the target gene, or to a sequence proximal thereto. Amplification can be performed by DNA polymerization reaction (such as PCR. RT-PCR) comprising a polymerase and the template nucleic acid to generate the amplicon. The amplicon is detected by any available detection method, e.g., sequencing (e.g., next generation sequencing), hybridizing the amplicon to an array (or affixing the amplicon to an array and hybridizing probes to it), digesting the amplicon with a restriction enzyme (e.g., RFLP), real-time PCR analysis, single nucleotide extension, allele-specific hybridization, or the like. Genotyping can also be performed by other known techniques, such as using primer mass extension and MALDI-TOF mass spectrum (MS) analysis, such as the MassEXTEND methodology of Sequenom, San Diego, Calif. In certain embodiments, primers for amplification are located on a chip. Amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. In certain embodiments, the method further comprises cleaving the amplified nucleic acid. Other methods for detecting the biomarkers herein are understood in the field and applicable to embodiments herein.

In some embodiments, one or more additional steps are taken upon identifying a subject as having an elevated risk of developing asthma or another condition. In some embodiments, methods further comprise a subsequent step of administering a treatment (e.g., therapeutic) or prophylactic, such as inhaled corticosteroids (fluticasone propionate (FLOVENT HFA, FLOVENT DISKUS, XHANCE), budesonide (PULMICORT FLEXHALER, PULMICORT RESPULES, RHINOCORT), ciclesonide (ALVESCO), beclomethasone (QVAR REDIHALER), mometasone (ASMANEX HFA, ASMANEX TWISTHALER) and fluticasone furoate (ARNUITY ELLIPTA), etc.), leukotriene modifiers (e.g., montelukast (SINGULAIR), zafirlukast (ACCOLATE), zileuton (ZYFLO), etc.), combination inhalers (e.g., fluticasone-salmeterol (ADVAIR HFA, AIRDUO DIGIHALER), budesonide-formoterol (SYMBICORT), formoterol-mometasone (DULERA) and fluticasone furoate-vilanterol (BREO ELLIPTA), theophylline (THEO-24, ELIXOPHYLLIN, THEOCHRON), etc.), bronchodilators (e.g., albuterol (PROAIR HFA. VENTOLIN HFA) and levalbuterol (XOPENEX. XOPENEX HFA), etc.), anticholinergic agents (e.g., ipratropium (ATROVENT HFA), tiotropium (SPIRIVA, SPIRIVA RESPIMAT), etc.), corticosteroids (e.g., prednisone (PREDNISONE INTENSOL, RAYOS) and methylprednisolone (MEDROL, DEPO-MEDROL, SOLU-MEDROL), etc.), etc. In some embodiments, methods further comprise cessation or avoidance of a treatment or activity that increases the risk of asthma, for example, cessation of smoking, avoidance of allergens, etc. In some embodiments, methods further comprise additional monitoring, such as spirometry, peak flow measurement, methacholine challenge, chest x-ray or other imaging, allergy testing, nitric oxide testing, detecting/quantifying eosinophils in sputum, testing for exercise and cold-induced asthma, etc. In some embodiments, methods further comprise a subsequent step of screening said subject for comorbidities. In some embodiments, methods further comprise generating a report indicating the presence/absence of the biomarkers tested, a risk score generated, an elevated or reduced risk (e.g., of AOA, of COA, etc.), and/or steps to be taken.

Some embodiments herein comprise the treatment of prevention of a disease/condition by editing genes that contain, for example, polymorphisms that lead to an increased risk for the development of the disease/condition (e.g., asthma (e.g., AOA, COA, etc.), autoimmune diseases, cancers, allergies, etc.). In some embodiments, expression of the polymorphisms herein is inhibited by modifying the polymorphic sequence in target cells. In some embodiments, the alteration of the polymorphic sequence is carried out using one or more DNA-binding nucleic acids, such as alteration via an RNA-guided endonuclease (RGEN). In some embodiments, the genetic alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for the target sequence (e.g., a sequence containing a polymorphism herein) and fixed tracrRNA) are introduced into the cell. In general, target sites at the 5′ end of the gRNA target the Cas nuclease to the target site using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., sequence containing a polymorphism herein). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression (e.g., to inhibit expression of the polymorphism). In some embodiments, the CRISPR system is used to alter the polymorphic sequence and/or inhibit expression of the polymorphism.

In some embodiments, nucleic acids encoding the polypeptides and/or fusions are inserted into the genetic material of a host using a CRISPR/Cas9 system. CRISPRs are DNA loci comprising short repetitions of base sequences. Each repetition is followed by short segments of “spacer DNA” from previous exposures to a virus. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. The CRISPR/Cas system may be used for gene editing. By delivering the Cas9 protein and appropriate guide RNAs into a cell, the organism's genome can be cut at any desired location. Methods for using CRISPR/Cas9 systems, and other systems, for insertion of a gene into a host cell to produce an engineered cell are described in, for example, U.S. Pub. No. 20180049412: herein incorporated by reference in its entirety. In some embodiments, the CRISPR/Cas system is used to “correct” polymorphisms described herein that are causative of an increased risk of asthma, for example: (1) one or more SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and rs9274660: (3) presence/absence of HLA-DQA1*0301: (3) the presence/absence of alanine (risk) or serine (protection) at position 31 of HLA-C: etc.

In some embodiments, asthma treatment/prevention methods described herein comprise administration (or co-administration with one or more additional therapies/therapeutics) of one or more inhibitors of the activity of HLA-DQA1*0301, HLA-DQA2, HLA-DQB2, and/or HLA-C (31A). In some embodiments, the inhibitor is administered to render lung epithelial cells less susceptible to the development of asthma. Any molecular or macromolecular entities or agents that target the aforementioned targets may find use in embodiments described herein. Inhibitors may be small molecules, peptides, polypeptides, proteins, nucleic acids, antibodies, antibody fragments, etc. In some embodiments, agents prevent a peptide displayed by and HLA from being recognized and/or being bound by T cells or other immunogenic agents.

In some embodiments, antibodies targeting HLA-DQA1*0301, HLA-DQA2, HLA-DQB2, and/or HLA-C (31A) are provided. In some embodiments, such antibodies or antibody fragments inhibit the activity of HLA-DQA1*0301, HLA-DQA2, HLA-DQB2, and/or HLA-C (31A). In some embodiments, such antibodies or antibody fragments inhibit the binding of T cells or other immune agents to HLA-DQA1*0301, HLA-DQA2, HLA-DQB2, and/or HLA-C (31A). Such antibodies may be naked or may be conjugated to a functional moiety (e.g., drug, toxin, effector moiety, etc.). In some embodiments, an antibody is a neutralizing antibody, a monoclonal antibody, a humanized antibody, and/or an antibody fragment.

In some embodiments, peptides, polypeptides, or small molecules that mimic peptides presented by HLA-DQA1*0301, HLA-DQA2, HLA-DQB2, and/or HLA-C (31A), and thereby inhibit binding of T cells or other immune agents to HLA-DQA1*0301, HLA-DQA2, HLA-DQB2, and/or HLA-C (31A) are provided.

In some embodiments, HLA-DQA1*0301, HLA-DQA2, HLA-DQB2, and/or HLA-C (31A) inhibitors are administered to a subject by any suitable method (e.g., intravenous, oral, inhaled, topical, nasal, etc.). In some embodiments, an inhibitor is administered to the lung epithelial cells of a subject (directly or indirectly).

In some embodiments, asthma treatment/prevention methods described herein comprise administration (or co-administration with one or more additional therapies/therapeutics) of one or more inhibitors of the expression of HLA-DQA1*0301, HLA-DQA2, HLA-DQB2, and/or HLA-C (31A). In some embodiments, a nucleic acid is used to modulate (e.g., inhibit) expression of HLA-DQA1*0301, HLA-DQA2, HLA-DQB2, and/or HLA-C (31A).

In some embodiments a small interfering RNA (siRNA) is designed to target and degrade a nucleic acid encoding HLA-DQA1*0301, HLA-DQA2, HLA-DQB2, and/or HLA-C (31A). siRNAs are double-stranded RNA molecules of 20-25 nucleotides in length. While not limited in their features, typically an siRNA is 21 nucleotides long and has 2-nt 3′ overhangs on both ends. Each strand has a 5′ phosphate group and a 3′ hydroxyl group. In vivo, this structure is the result of processing by Dicer, an enzyme that converts either long dsRNAs or small hairpin RNAs (shRNAs) into siRNAs. However, siRNAs can also be synthesized and exogenously introduced into cells to bring about the specific knockdown of a gene of interest. Essentially any gene of which the sequence is known can be targeted based on sequence complementarity with an appropriately tailored siRNA. For example, those of ordinary skill in the art can synthesize an siRNA (see, e.g., Elbashir, et al., Nature 411: 494 (2001): Elbashir, et al. Genes Dev 15:188 (2001): Tuschl T, et al., Genes Dev 13:3191 (1999)).

In some embodiments, RNAi is utilized to inhibit HLA-DQA1*0301, HLA-DQA2, HLA-DQB2, and/or HLA-C (31A). RNAi represents an evolutionarily conserved cellular defense for controlling the expression of foreign genes in most eukaryotes, including humans. RNAi is typically triggered by double-stranded RNA (dsRNA) and causes sequence-specific degradation of single-stranded target RNAs (e.g., an mRNA). The mediators of mRNA degradation are small interfering RNAs (siRNAs), which are normally produced from long dsRNA by enzymatic cleavage in the cell. siRNAs are generally approximately twenty-one nucleotides in length (e.g. 21-23 nucleotides in length) and have a base-paired structure characterized by two-nucleotide 3′ overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, an RNase III enzyme (e.g., Dicer) converts the longer dsRNA into 21-23 nt double-stranded siRNA fragments. In some embodiments, RNAi oligonucleotides are designed to target HLA-DQA1*0301, HLA-DQA2, HLA-DQB2, and/or HLA-C (31A).

In other embodiments, shRNA techniques (See e.g., 20080025958, herein incorporated by reference in its entirety) are utilized to modulate (e.g., inhibit) expression of HLA-DQA1*0301, HLA-DQA2, HLA-DQB2, and/or HLA-C (31A). A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA that is bound to it. shRNA is transcribed by RNA polymerase III.

In some embodiments, the technology described herein uses antisense nucleic acid (e.g., an antisense DNA oligo, an antisense RNA oligo) to modulate (e.g., inhibit) the expression of HLA-DQA1*0301, HLA-DQA2, HLA-DQB2, and/or HLA-C (31A). For example, in some embodiments, expression modulated (e.g., inhibited) using antisense compounds that specifically hybridize with one or more nucleic acids encoding HLA-DQA1*0301, HLA-DQA2, HLA-DQB2, and/or HLA-C (31A). The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of HLA-DQA1*0301, HLA-DQA2, HLA-DQB2, and/or HLA-C (31A).

Further molecules effecting RNAi (and useful herein for the inhibition of expression of polymorphic sequences) include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of target expression after introduction into target cells. In some embodiments, provided herein are miRNA molecules that target and inhibit the expression (e.g., knock down) of the sequences herein.

Morpholinos (or morpholino oligonucleotides) are synthetic nucleic acid molecules having a length of about 20 to 30 nucleotides and, typically about 25 nucleotides. Morpholinos bind to complementary sequences of target transcripts by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knock down) of the target sequence.

A ribozyme (ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors oligonucleotides of the target sequences described herein.

In some embodiments, provided herein are methods and compositions for the introduction of a gene encoding a protein or peptide that inhibits (or enhances) HLA-DQA2 and or HLA-DQB2 levels or function. In some embodiments, an mRNA or other transgene is administered to a subject and the mRNA or transgene is expressed within the subject.

### Experimental

Using Bayesian approaches for fine-mapping GWAS results and fine-mapping expression quantitative trait loci (eQTLs) of HLA region genes in cell types relevant to asthma, experiments were conducted during development of embodiments herein to identify causal variants in the HLA class I and class II regions for COA and AOA, and to examine the hypotheses that (1) the causal variants at the HLA locus include those that are both shared and distinct to COA and AOA, and (2) that some causal variants exert their effects on asthma risk by modifying the expression of HLA genes while others alter protein function by changing the amino acid sequence in the functional domains of the protein.

### Results

### HLA Allele and Amino Acid Associations

Studies focused our studies on the 9,432 COA cases, 21,556 AOA cases, and 318,167 shared non-asthma controls used in Pividori, et al. (Ref. 3: incorporated by reference in its entirety). Because the GWASs for COA and AOA included only SNPs, the imputed 4-digit HLA alleles provided were first considered, and associations between the HLA-A, -B, -C, -DQA1, -DQB1, and -DRB1 alleles with COA and AOA were examined. Out of 78 HLA alleles at 6 loci, 19 were associated with COA and 14 with AOA (p<5×10⁻⁸. Tables 1-2). Using the same criteria as Pividori et al. for assigning associations as specific to COA or AOA or shared, one allele (HLA-C*05:01) was COA specific, 3 alleles (HLA-DQA1*01:02, HLA-DQB1*06:02. HLA-DRB1*15:01) were AOA specific: the remaining 20 associations were shared.

TABLE 1A
COA HLA Allele Associations.

| | Childhood-Onset Asthma | | | | |
|---|---|---|---|---|---|
| HLA allele | p-value | beta | SE | OR | 95% OR CI |
| B*08:01 | 3.21 × 10⁻⁶ | 0.10 | 0.02 | 1.10 | 1.06-1.14 |
| B*35:01 | 5.20 × 10⁻¹³ | −0.29 | 0.04 | 0.75 | 0.69-0.81 |
| B*44:02 | 4.06 × 10⁻¹⁰ | 0.14 | 0.02 | 1.15 | 1.10-1.20 |
| C*04:01 | 6.16 × 10⁻¹² | −0.20 | 0.03 | 0.82 | 0.77-0.87 |
| C*05:01 | 6.35 × 10⁻⁹ | 0.13 | 0.02 | 1.14 | 1.09-1.19 |
| DQA1*01:01 | 1.98 × 10⁻³⁵ | −0.29 | 0.02 | 0.75 | 0.71-0.78 |
| DQA1*01:02 | 1.84 × 10⁻¹ | −0.03 | 0.02 | 0.98 | 0.94-1.01 |
| DQA1*01:03 | 5.08 × 10⁻¹⁰ | −0.22 | 0.04 | 0.80 | 0.75-0.86 |
| DQA1*03:01 | 7.26 × 10⁻¹⁷ | 0.15 | 0.02 | 1.16 | 1.12-1.20 |
| DQA1*05:01 | 2.01 × 10⁻¹⁴ | 0.13 | 0.02 | 1.14 | 1.10-1.18 |
| DQB1*02:01 | 2.93 × 10⁻⁹ | 0.12 | 0.02 | 1.13 | 1.08-1.17 |
| DQB1*03:01 | 4.15 × 10⁻¹⁸ | 0.16 | 0.02 | 1.18 | 1.13-1.22 |
| DQB1*03:02 | 4.65 × 10⁻⁴ | 0.08 | 0.02 | 1.09 | 1.04-1.14 |
| DQB1*05:01 | 4.18 × 10⁻²⁵ | −0.26 | 0.02 | 0.77 | 0.76-0.81 |
| DQB1*05:03 | 1.35 × 10⁻¹¹ | −0.41 | 0.06 | 0.66 | 0.59-0.75 |
| DQB1*06:02 | 2.33 × 10⁻² | 0.05 | 0.02 | 1.05 | 1.01-1.09 |
| DQB1*06:03 | 9.88 × 10⁻⁹ | −0.21 | 0.04 | 0.81 | 0.76-0.87 |
| DRB1*01:01 | 3.65 × 10⁻²³ | −0.28 | 0.03 | 0.75 | 0.71-0.80 |
| DRB1*03:01 | 3.84 × 10⁻¹⁰ | 0.13 | 0.02 | 1.13 | 1.09-1.18 |
| DRB1*04:01 | 8.56 × 10⁻²² | 0.21 | 0.02 | 1.24 | 1.18-1.29 |
| DRB1*11:01 | 4.29 × 10⁻¹⁰ | 0.24 | 0.04 | 1.27 | 1.18-1.37 |
| DRB1*13:01 | 1.96 × 10⁻⁸ | −0.21 | 0.04 | 0.81 | 0.76-0.87 |
| DRB1*14:01 | 4.54 × 10⁻¹⁰ | −0.40 | 0.06 | 0.67 | 0.59-0.76 |
| DRB1*15:01 | 5.51 × 10⁻² | 0.04 | 0.02 | 1.04 | 0.99-1.08 |

Associations for significant HLA alleles (p < 5 × 10 − 8; bolded) in COA.
SE: standard error.
OR: odds ratio.
CI: confidence interval.

TABLE 1B
AOA HLA Allele Associations.
Adult-Onset Asthma

| HLA allele | p-value | beta | SE | OR | 95% OR CI |
|---|---|---|---|---|---|
| B*08:01 | 9.19 × 10⁻¹⁰ | 0.08 | 0.01 | 1.09 | 1.06-1.12 |
| B*35:01 | 2.22 × 10⁻⁶ | −0.12 | 0.02 | 0.89 | 0.85-0.93 |
| B*44:02 | 2.89 × 10⁻³ | 0.05 | 0.02 | 1.05 | 1.02-1.08 |
| C*04:01 | 5.39 × 10⁻⁶ | −0.08 | 0.02 | 0.92 | 0.89-0.95 |
| C*05:01 | 8.18 × 10⁻² | 0.03 | 0.02 | 1.03 | 0.99-1.06 |
| DQA1*01:01 | 1.89 × 10⁻¹⁶ | −0.12 | 0.01 | 0.89 | 0.86-0.91 |
| DQA1*01:02 | 1.23 × 10⁻¹³ | −0.10 | 0.01 | 0.91 | 0.89-0.93 |
| DQA1*01:03 | 1.45 × 10⁻⁸ | −0.13 | 0.02 | 0.88 | 0.84-0.92 |
| DQA1*03:01 | 6.91 × 10⁻⁴⁷ | 0.17 | 0.01 | 1.19 | 1.16-1.22 |
| DQA1*05:01 | 2.92 × 10⁻³ | 0.04 | 0.01 | 1.04 | 1.01-1.06 |
| DQB1*02:01 | 8.52 × 10⁻⁵ | 0.05 | 0.01 | 1.06 | 1.03-1.08 |
| DQB1*03:01 | 1.82 × 10⁻⁹ | 0.08 | 0.01 | 1.08 | 1.05-1.11 |
| DQB1*03:02 | 5.68 × 10⁻²⁵ | 0.16 | 0.02 | 1.17 | 1.14-1.21 |
| DQB1*05:01 | 1.73 × 10⁻¹³ | −0.12 | 0.02 | 0.89 | 0.86-0.92 |
| DQB1*05:03 | 5.07 × 10⁻³ | −0.10 | 0.04 | 0.905 | 0.84-0.97 |
| DQB1*06:02 | 1.21 × 10⁻⁸ | −0.08 | 0.01 | 0.92 | 0.89-0.95 |
| DQB1*06:03 | 5.10 × 10⁻¹⁰ | −0.15 | 0.02 | 0.86 | 0.82-0.90 |
| DRB1*01:01 | 2.41 × 10⁻¹¹ | −0.12 | 0.02 | 0.89 | 0.86-0.92 |
| DRB1*03:01 | 1.33 × 10⁻⁴ | 0.05 | 0.01 | 1.05 | 1.03-1.08 |
| DRB1*04:01 | 1.03 × 10⁻³⁴ | 0.19 | 0.02 | 1.20 | 1.17-1.24 |
| DRB1*11:01 | 2.58 × 10⁻² | 0.06 | 0.03 | 1.06 | 1.01-1.12 |
| DRB1*13:01 | 1.69 × 10⁻⁹ | −0.15 | 0.02 | 0.87 | 0.82-0.91 |
| DRB1*14:01 | 1.22 × 10⁻² | −0.09 | 0.04 | 0.91 | 0.85-0.98 |
| DRB1*15:01 | 3.56 × 10⁻⁹ | −0.09 | 0.01 | 0.92 | 0.89-0.94 |

Associations for significant HLA alleles (p < 5 × 10 − 8; bolded) in AOA.
SE: standard error.
OR: odds ratio.
CI: confidence interval.

TABLE 2A
HLA Allele Associations for COA. Results
are shown for each HLA alleles (freq >1%).

| | Childhood-Onset Asthma | | | | |
|---|---|---|---|---|---|
| HLA allele | p-value | OR | 95% CI | beta | SE |
| A*01:01 | 5.23E−02 | 1.037 | 0.999-1.075 | 0.036 | 0.019 |
| A*02:01 | 6.05E−03 | 0.955 | 0.923-0.986 | −0.046 | 0.017 |
| A*03:01 | 1.86E−01 | 0.972 | 0.932-1.013 | −0.028 | 0.021 |
| A*11:01 | 8.79E−01 | 1.005 | 0.945-1.067 | 0.005 | 0.031 |
| A*23:01 | 7.39E−01 | 1.019 | 0.911-1.139 | 0.019 | 0.057 |
| A*24:02 | 9.36E−01 | 1.002 | 0.947-1.060 | 0.002 | 0.029 |
| A*25:01 | 3.98E−02 | 1.125 | 1.005-1.258 | 0.118 | 0.057 |
| A*26:01 | 9.75E−01 | 1.002 | 0.899-1.115 | 0.002 | 0.055 |
| A*29:02 | 5.80E−04 | 1.128 | 1.053-1.208 | 0.120 | 0.035 |
| A*30:01 | 1.17E−01 | 1.116 | 0.972-1.281 | 0.110 | 0.070 |
| A*31:01 | 5.71E−01 | 1.026 | 0.938-1.121 | 0.026 | 0.045 |
| A*32:01 | 7.38E−01 | 1.014 | 0.936-1.096 | 0.013 | 0.040 |
| A*68:01 | 3.83E−02 | 0.911 | 0.834-0.995 | −0.093 | 0.045 |
| B*07:02 | 1.34E−01 | 0.969 | 0.929-1.009 | −0.032 | 0.021 |
| B*08:01 | 3.21E−06 | 1.100 | 1.056-1.144 | 0.095 | 0.020 |
| B*13:02 | 9.41E−01 | 1.004 | 0.903-1.115 | 0.004 | 0.054 |
| B*14:01 | 5.26E−01 | 0.956 | 0.831-1.099 | −0.045 | 0.071 |
| B*14:02 | 1.98E−01 | 0.940 | 0.854-1.033 | −0.062 | 0.048 |
| B*15:01 | 1.13E−03 | 1.101 | 1.039-1.167 | 0.097 | 0.030 |
| B*18:01 | 6.83E−01 | 1.016 | 0.940-1.097 | 0.016 | 0.039 |
| B*27:05 | 5.55E−02 | 0.927 | 0.857-1.001 | −0.076 | 0.040 |
| B*35:01 | 5.20E−13 | 0.749 | 0.692-0.810 | −0.289 | 0.040 |
| B*37:01 | 5.27E−01 | 0.960 | 0.844-1.090 | −0.041 | 0.065 |
| B*40:01 | 2.63E−01 | 0.964 | 0.904-1.027 | −0.037 | 0.033 |
| B*44:02 | 4.06E−10 | 1.150 | 1.100-1.201 | 0.140 | 0.022 |
| B*44:03 | 4.73E−05 | 1.131 | 1.065-1.199 | 0.123 | 0.030 |
| B*49:01 | 9.84E−01 | 0.999 | 0.870-1.145 | −0.001 | 0.070 |
| B*51:01 | 1.50E−01 | 0.943 | 0.870-1.021 | −0.059 | 0.041 |
| B*55:01 | 1.81E−01 | 0.927 | 0.829-1.035 | −0.076 | 0.057 |
| B*57:01 | 4.97E−04 | 0.869 | 0.802-0.940 | −0.141 | 0.040 |
| C*01:02 | 3.51E−06 | 0.812 | 0.743-0.886 | −0.208 | 0.045 |
| C*02:02 | 6.53E−01 | 1.018 | 0.942-1.099 | 0.018 | 0.039 |
| C*03:03 | 6.90E−01 | 1.013 | 0.951-1.078 | 0.013 | 0.032 |
| C*03:04 | 2.31E−01 | 1.033 | 0.979-1.088 | 0.032 | 0.027 |
| C*04:01 | 6.16E−12 | 0.819 | 0.774-0.867 | −0.199 | 0.029 |
| C*05:01 | 6.35E−09 | 1.138 | 1.089-1.189 | 0.129 | 0.022 |
| C*06:02 | 2.73E−02 | 0.943 | 0.895-0.993 | −0.058 | 0.026 |
| C*07:01 | 4.83E−04 | 1.069 | 1.029-1.109 | 0.067 | 0.019 |
| C*07:02 | 1.42E−01 | 0.970 | 0.932-1.010 | −0.030 | 0.021 |
| C*07:04 | 6.31E−03 | 1.156 | 1.041-1.283 | 0.145 | 0.053 |
| C*08:02 | 1.21E−01 | 0.939 | 0.866-1.016 | −0.063 | 0.041 |
| C*12:03 | 9.59E−02 | 0.926 | 0.846-1.013 | −0.077 | 0.046 |
| C*15:02 | 7.09E−01 | 0.979 | 0.873-1.095 | −0.022 | 0.058 |
| C*16:01 | 8.91E−06 | 1.163 | 1.087-1.242 | 0.151 | 0.034 |
| DQA1*01:01 | 1.98E−35 | 0.747 | 0.713-0.782 | −0.292 | 0.023 |
| DQA1*01:02 | 1.84E−01 | 0.975 | 0.939-1.012 | −0.025 | 0.019 |
| DQA1*01:03 | 5.08E−10 | 0.802 | 0.748-0.859 | −0.221 | 0.035 |
| DQA1*02:01 | 9.52E−01 | 0.999 | 0.958-1.040 | −0.001 | 0.021 |
| DQA1*03:01 | 7.26E−17 | 1.160 | 1.120-1.200 | 0.148 | 0.018 |
| DQA1*04:01 | 4.79E−02 | 0.896 | 0.802-0.998 | −0.110 | 0.056 |
| DQA1*05:01 | 2.01E−14 | 1.140 | 1.102-1.179 | 0.131 | 0.017 |
| DQB1*02:01 | 2.93E−09 | 1.126 | 1.082-1.171 | 0.119 | 0.020 |
| DQB1*02:02 | 2.84E−02 | 1.060 | 1.006-1.117 | 0.059 | 0.027 |
| DQB1*03:01 | 4.15E−18 | 1.175 | 1.132-1.218 | 0.161 | 0.019 |
| DQB1*03:02 | 4.65E−04 | 1.086 | 1.037-1.137 | 0.083 | 0.024 |
| DQB1*03:03 | 1.22E−03 | 0.894 | 0.834-0.956 | −0.113 | 0.035 |
| DQB1*04:02 | 4.19E−02 | 0.894 | 0.802-0.995 | −0.112 | 0.055 |
| DQB1*05:01 | 4.18E−25 | 0.772 | 0.735-0.810 | −0.259 | 0.025 |
| DQB1*05:03 | 1.35E−11 | 0.663 | 0.588-0.746 | −0.411 | 0.061 |
| DQB1*06:02 | 2.33E−02 | 1.049 | 1.006-1.092 | 0.048 | 0.021 |
| DQB1*06:03 | 9.88E−09 | 0.811 | 0.755-0.871 | −0.209 | 0.037 |
| DQB1*06:04 | 2.47E−07 | 0.771 | 0.697-0.850 | −0.261 | 0.051 |
| DQB1*06:09 | 7.20E−02 | 0.868 | 0.743-1.012 | −0.142 | 0.079 |
| DRB1*01:01 | 3.65E−23 | 0.754 | 0.712-0.797 | −0.283 | 0.029 |
| DRB1*01:03 | 6.56E−04 | 0.806 | 0.712-0.912 | −0.215 | 0.063 |
| DRB1*03:01 | 3.84E−10 | 1.133 | 1.089-1.178 | 0.125 | 0.020 |
| DRB1*04:01 | 8.56E−22 | 1.236 | 1.183-1.290 | 0.212 | 0.022 |
| DRB1*04:04 | 1.51E−01 | 1.055 | 0.980-1.133 | 0.053 | 0.037 |
| DRB1*07:01 | 9.29E−01 | 0.998 | 0.957-1.040 | −0.002 | 0.021 |
| DRB1*08:01 | 5.66E−02 | 0.895 | 0.798-1.003 | −0.111 | 0.058 |
| DRB1*09:01 | 8.66E−01 | 1.011 | 0.890-1.148 | 0.011 | 0.065 |
| DRB1*11:01 | 4.29E−10 | 1.270 | 1.178-1.368 | 0.239 | 0.038 |
| DRB1*11:04 | 2.96E−01 | 0.926 | 0.801-1.069 | −0.077 | 0.074 |
| DRB1*12:01 | 5.07E−01 | 0.959 | 0.847-1.085 | −0.042 | 0.063 |
| DRB1*13:01 | 1.96E−08 | 0.814 | 0.757-0.874 | −0.206 | 0.037 |
| DRB1*13:02 | 7.27E−08 | 0.795 | 0.731-0.864 | −0.230 | 0.043 |
| DRB1*14:01 | 4.54E−10 | 0.671 | 0.591-0.760 | −0.400 | 0.064 |
| DRB1*15:01 | 5.51E−02 | 1.041 | 0.999-1.084 | 0.040 | 0.021 |

Significant associations (p < 5 × 10\hat{ } − 8) with are shown in bold font.

TABLE 2B
HLA Allele Associations for AOA. Results
are shown for each HLA alleles (freq >1%).

| | Adult-Onset Asthma | | | | |
|---|---|---|---|---|---|
| HLA allele | p-value | OR | 95% CI | beta | SE |
| A*01:01 | 8.62E−03 | 1.033 | 1.008-1.059 | 0.033 | 0.013 |
| A*02:01 | 8.83E−01 | 0.998 | 0.976-1.020 | −0.002 | 0.011 |
| A*03:01 | 1.17E−03 | 0.954 | 0.927-0.981 | −0.047 | 0.014 |
| A*11:01 | 3.23E−02 | 0.955 | 0.916-0.996 | −0.046 | 0.021 |
| A*23:01 | 9.43E−01 | 1.003 | 0.929-1.081 | 0.003 | 0.039 |
| A*24:02 | 1.88E−01 | 1.025 | 0.987-1.064 | 0.025 | 0.019 |
| A*25:01 | 7.72E−01 | 1.012 | 0.934-1.095 | 0.012 | 0.040 |
| A*26:01 | 9.45E−01 | 1.003 | 0.932-1.078 | 0.003 | 0.037 |
| A*29:02 | 3.81E−01 | 1.022 | 0.973-1.072 | 0.022 | 0.025 |
| A*30:01 | 9.47E−01 | 1.003 | 0.910-1.106 | 0.003 | 0.050 |
| A*31:01 | 2.58E−04 | 1.114 | 1.051-1.180 | 0.108 | 0.030 |
| A*32:01 | 8.10E−01 | 1.007 | 0.954-1.061 | 0.007 | 0.027 |
| A*68:01 | 5.26E−01 | 0.982 | 0.926-1.039 | −0.019 | 0.029 |
| B*07:02 | 7.98E−07 | 0.932 | 0.905-0.958 | −0.071 | 0.014 |
| B*08:01 | 9.19E−10 | 1.088 | 1.059-1.118 | 0.085 | 0.014 |
| B*13:02 | 1.90E−01 | 1.048 | 0.977-1.123 | 0.047 | 0.036 |
| B*14:01 | 2.25E−01 | 0.943 | 0.858-1.036 | −0.058 | 0.048 |
| B*14:02 | 5.82E−03 | 0.913 | 0.856-0.974 | −0.091 | 0.033 |
| B*15:01 | 3.87E−07 | 1.107 | 1.064-1.151 | 0.102 | 0.020 |
| B*18:01 | 1.58E−01 | 0.962 | 0.912-1.015 | −0.038 | 0.027 |
| B*27:05 | 9.12E−01 | 0.997 | 0.947-1.048 | −0.003 | 0.026 |
| B*35:01 | 2.22E−06 | 0.889 | 0.846-0.933 | −0.118 | 0.025 |
| B*37:01 | 2.47E−01 | 0.950 | 0.871-1.035 | −0.051 | 0.044 |
| B*40:01 | 6.73E−01 | 0.991 | 0.949-1.034 | −0.009 | 0.022 |
| B*44:02 | 2.89E−03 | 1.048 | 1.016-1.080 | 0.046 | 0.016 |
| B*44:03 | 5.68E−02 | 1.041 | 0.998-1.084 | 0.040 | 0.021 |
| B*49:01 | 7.44E−01 | 0.985 | 0.897-1.080 | −0.015 | 0.047 |
| B*51:01 | 3.20E−01 | 1.027 | 0.974-1.081 | 0.026 | 0.027 |
| B*55:01 | 2.12E−01 | 0.954 | 0.885-1.027 | −0.047 | 0.038 |
| B*57:01 | 1.31E−05 | 0.890 | 0.843-0.937 | −0.117 | 0.027 |
| C*01:02 | 9.63E−02 | 0.955 | 0.903-1.008 | −0.047 | 0.028 |
| C*02:02 | 1.39E−01 | 1.040 | 0.987-1.094 | 0.039 | 0.026 |
| C*03:03 | 9.66E−03 | 1.057 | 1.013-1.101 | 0.055 | 0.021 |
| C*03:04 | 6.03E−02 | 1.035 | 0.998-1.072 | 0.034 | 0.018 |
| C*04:01 | 5.39E−06 | 0.919 | 0.885-0.952 | −0.085 | 0.019 |
| C*05:01 | 8.18E−02 | 1.028 | 0.996-1.059 | 0.027 | 0.016 |
| C*06:02 | 9.28E−03 | 0.955 | 0.922-0.988 | −0.046 | 0.018 |
| C*07:01 | 1.51E−06 | 1.064 | 1.037-1.091 | 0.062 | 0.013 |
| C*07:02 | 4.81E−06 | 0.938 | 0.912-0.964 | −0.064 | 0.014 |
| C*07:04 | 1.43E−01 | 1.056 | 0.981-1.136 | 0.055 | 0.037 |
| C*08:02 | 5.35E−03 | 0.926 | 0.877-0.977 | −0.077 | 0.028 |
| C*12:03 | 5.33E−01 | 0.981 | 0.924-1.041 | −0.019 | 0.030 |
| C*15:02 | 2.21E−01 | 1.047 | 0.972-1.128 | 0.046 | 0.038 |
| C*16:01 | 1.53E−02 | 1.059 | 1.011-1.109 | 0.058 | 0.024 |
| DQA1*01:01 | 1.89E−16 | 0.885 | 0.859-0.911 | −0.122 | 0.015 |
| DQA1*01:02 | 1.23E−13 | 0.908 | 0.885-0.931 | −0.097 | 0.013 |
| DQA1*01:03 | 1.45E−08 | 0.878 | 0.839-0.918 | −0.130 | 0.023 |
| DQA1*02:01 | 9.54E−01 | 1.001 | 0.973-1.029 | 0.001 | 0.014 |
| DQA1*03:01 | 6.91E−47 | 1.187 | 1.159-1.215 | 0.172 | 0.012 |
| DQA1*04:01 | 4.46E−01 | 0.973 | 0.905-1.044 | −0.028 | 0.036 |
| DQA1*05:01 | 2.92E−03 | 1.036 | 1.012-1.060 | 0.035 | 0.012 |
| DQB1*02:01 | 8.52E−05 | 1.056 | 1.027-1.084 | 0.054 | 0.014 |
| DQB1*02:02 | 4.94E−02 | 1.036 | 1.000-1.073 | 0.036 | 0.018 |
| DQB1*03:01 | 1.82E−09 | 1.080 | 1.053-1.108 | 0.077 | 0.013 |
| DQB1*03:02 | 5.68E−25 | 1.174 | 1.138-1.210 | 0.161 | 0.016 |
| DQB1*03:03 | 2.06E−01 | 0.972 | 0.929-1.015 | −0.029 | 0.023 |
| DQB1*04:02 | 2.05E−01 | 0.955 | 0.889-1.025 | −0.046 | 0.036 |
| DQB1*05:01 | 1.73E−13 | 0.890 | 0.862-0.917 | −0.117 | 0.016 |
| DQB1*05:03 | 5.07E−03 | 0.905 | 0.844-0.970 | −0.099 | 0.036 |
| DQB1*06:02 | 1.21E−08 | 0.920 | 0.893-0.946 | −0.084 | 0.015 |
| DQB1*06:03 | 5.10E−10 | 0.862 | 0.821-0.902 | −0.149 | 0.024 |
| DQB1*06:04 | 4.26E−05 | 0.877 | 0.823-0.934 | −0.131 | 0.032 |
| DQB1*06:09 | 3.22E−01 | 0.951 | 0.860-1.050 | −0.050 | 0.051 |
| DRB1*01:01 | 2.41E−11 | 0.887 | 0.856-0.918 | −0.120 | 0.018 |
| DRB1*01:03 | 3.55E−01 | 0.965 | 0.893-1.041 | −0.036 | 0.039 |
| DRB1*03:01 | 1.33E−04 | 1.054 | 1.026-1.083 | 0.053 | 0.014 |
| DRB1*04:01 | 1.03E−34 | 1.203 | 1.168-1.239 | 0.185 | 0.015 |
| DRB1*04:04 | 1.01E−05 | 1.114 | 1.061-1.168 | 0.108 | 0.024 |
| DRB1*07:01 | 9.37E−01 | 0.999 | 0.971-1.026 | −0.001 | 0.014 |
| DRB1*08:01 | 6.42E−01 | 0.983 | 0.912-1.058 | −0.018 | 0.038 |
| DRB1*09:01 | 3.00E−04 | 1.161 | 1.070-1.258 | 0.149 | 0.041 |
| DRB1*11:01 | 2.58E−02 | 1.064 | 1.007-1.123 | 0.062 | 0.028 |
| DRB1*11:04 | 6.64E−01 | 0.979 | 0.890-1.077 | −0.021 | 0.049 |
| DRB1*12:01 | 1.14E−04 | 0.840 | 0.768-0.917 | −0.175 | 0.045 |
| DRB1*13:01 | 1.69E−09 | 0.865 | 0.824-0.906 | −0.145 | 0.024 |
| DRB1*13:02 | 2.23E−05 | 0.891 | 0.844-0.939 | −0.115 | 0.027 |
| DRB1*14:01 | 1.22E−02 | 0.910 | 0.845-0.979 | −0.094 | 0.038 |
| DRB1*15:01 | 3.56E−09 | 0.917 | 0.891-0.944 | −0.086 | 0.015 |

Significant associations (p < 5 × 10\hat{ } − 8) with are shown in bold font.

741 amino acid polymorphisms across the 6 HLA loci were then tested for association with COA and AOA. 188 amino acid polymorphisms were associated with COA and 152 with AOA (p<5×10⁻⁸). Three were COA-specific and 14 were AOA-specific, and the remaining 197 associations were shared. The most significant class I and class II amino acid polymorphisms were HLA-C Ser11 (p=3.12×10⁻¹⁹; OR=0.81, 95% CI 0.77-0.84) and HLA-DQB1 His30 (p=6.04×10⁻⁵⁶; OR=0.74, CI 0.71-0.77) with COA, and HLA-C Tyr99 (p=1.78×10⁻¹²; OR=1.08, CI 1.06-1.11) and HLA-DQB1 Arg55 (p=4.50×10⁻⁴⁹; OR=0.86, CI 0.84-0.88) with AOA.

P-values were overall more significant and estimated ORs were larger for class II compared to class I HLA alleles and amino acid polymorphisms, and the magnitude of the ORs were generally larger for COA compared to AOA, both consistent with the GWAS results. To fully capture variation at the HLA region, the genotypes for 19,499 HLA alleles, amino acid polymorphisms and SNPs we combined, and fine-mapping was performed on the combined set of variants separately in the HLA class I and class II regions, with the goal of identifying causal variation for COA and AOA.

### Fine-Mapping the HLA Class I Region

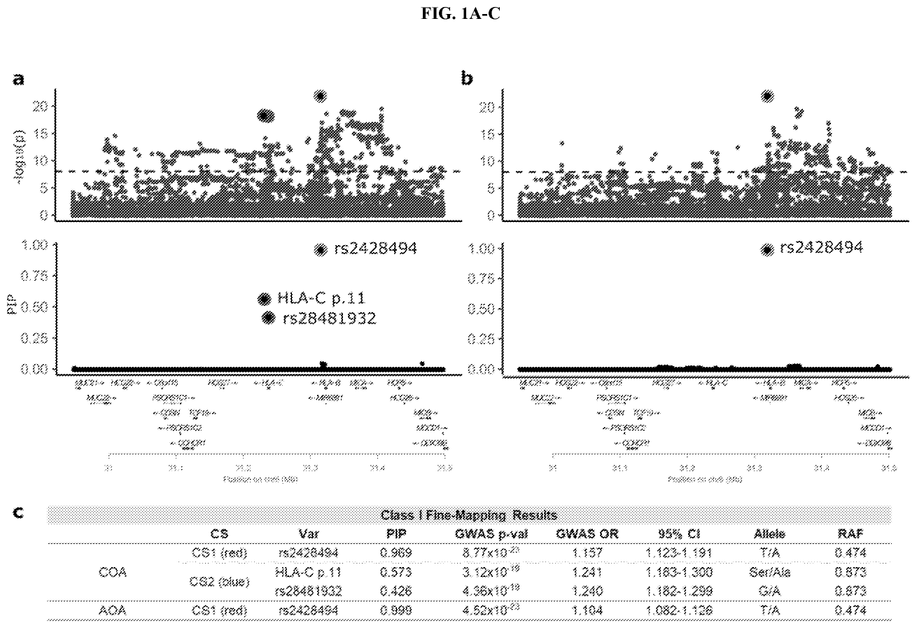

The Bayesian regression method Sum of Single Effects (SuSiE) (Ref. 14; incorporated by reference in its entirety) was used to perform genetic fine-mapping of the 9,021 combined variants at the class I locus, using the locus boundaries defined in Pividori, et al, and including genotypes for SNPs spanning the region and HLA-A, HLA-B, and HLA-C alleles and amino acid polymorphisms. SuSiE reports credible sets (CSs)—sets of variants that are as small as possible while containing at least one causal variant with high probability. Fine-mapping the COA class I locus identified 2 CSs, indicating the presence of 2 distinct associations with COA in the class I region. Credible set 1 (CS1) consisted of a single variant, and CS2 consisted of 2 variants (FIG. 1a). The CS1 (red point, FIG. 1a) SNP (rs2428494) is in an intron of HLA-B, and its posterior inclusion probability (PIP) is 0.97. This was the lead SNP in both the COA and AOA GWASs at the HLA class I locus. In CS2 (blue points, FIG. 1a), the probability was nearly equally divided between 2 highly correlated variants (LD r²=0.99, calculated from our data). One was a SNP (rs28481932) upstream of HLA-C and one was an amino acid polymorphism in HLA-C (p.11 Ala/Ser). The risk amino acid (alanine) is on all HLA-C*02, *03, *05, *06, *07, *08, *12, *15, *16, *17, and *18 alleles in the data (including rare alleles), and the protective amino acid (serine) is on the HLA-C*01:02, *04:01, *04:07, *14:02, and *14:03 alleles. The PIPs for rs28481932 and HLA-C p.11 were 0.43 and 0.57, respectively, slightly favoring HLA-C p.11 over rs28481932 as the causal variant in CS2.

Only one CS (containing rs2428494) was identified for AOA at the class I locus (FIG. 1b). These results indicate that rs2428494 is a shared causal SNP for COA and AOA (FIG. 1c) and HLA-C p.11 or rs28481932 is a causal variant for COA, or each tags untyped or rare causal variation in LD with the candidate variant.

### Fine-Mapping the HLA Class II Region

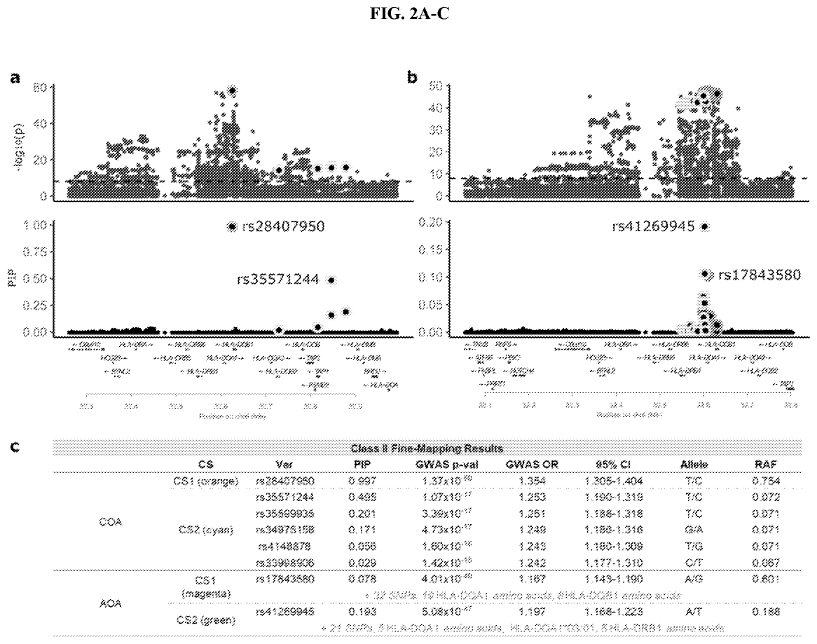

10,428 combined variants at the class II region were used for fine-mapping, including genotypes for SNPs across this region and HLA-DRB1. HLA-DQB1, and HLA-DQA1 alleles and amino acid polymorphisms. Two CSs for COA were identified (FIG. 2a). CS1 (orange point) contained one SNP (rs28407950) located downstream of HLA-DQB1. This was the lead SNP in the HLA class II region for COA. CS2 (cyan points) contained 5 SNPs spanning 152 kb. The SNP with the highest PIP (rs35571244) was located at the proximal end of the class II region upstream of TAP1. The minimum r² between all variants in CS2 was 0.79 (median r²=0.99). No HLA alleles or amino acid polymorphisms were included in either of the COA CSs.

Two CSs were also identified for AOA, but neither included SNPs in the COA CSs (FIG. 2b). CS1 (magenta points) contained 60 variants: 33 SNPs, 19 HLA-DQA1 amino acid polymorphisms, and 8 HLA-DQB1 amino acid polymorphisms. The minimum r² between all variants in CS1 was 0.94 (median r²=0.99), spanning 32.1 kb across the HLA-DQA1 and HLA-DQB1 genes. The variant with the highest PIP in CS1 was a SNP (rs17843580) located downstream of HLA-DQA1, which was the lead SNP in the AOA GWAS. CS2 (green) spanned 54.6 kb from HLA-DRB5 to HLA-DQA1, and included 33 variants: 22 SNPs, 5 HLA-DQA1 amino acid polymorphisms, one HLA-DQA1 allele (HLA-DQA1*03:01), and 5 HLA-DRB1 amino acid polymorphisms. The minimum r² between all CS2 variants was 0.88 (median r²=0.96). The variant with the highest PIP was a SNP (rs41269945) located between HLA-DQA1 and HLA-DQB1. Five perfectly correlated HLA-DQA1 amino acids had the highest PIPs among the amino acids: Thr26, Gln47, Arg56, Val76, and Thr187, which define HLA-DQA1*03 alleles (*03:01, *03:02, and *03:03 in the data).

Overall, the variation in the class II region was not shared between COA and AOA (FIG. 2c, Table 3). The variants for COA were comprised entirely of SNPs in non-coding regions, whereas the candidate variants for AOA included both non-coding SNPs and protein coding (amino acid) variation in the HLA-DQB1, HLA-DQA1, and HLA-DRB1 class II genes.

TABLE 3
SuSiE Credible Set Results
Median r2 reported between all variants within the CS. Posterior Inclusion
Probability (PIP) reported for each variant within the CS. P-value, odds
ratio (OR) and confidence interval (CI) reported for the variant's association
for that specific group (childhood-onset asthma or adult-onset asthma).

| | | | Median | | | | | | | | |
|---|---|---|---|---|---|---|---|---|---|---|---|
| Region | Group | CS | r2 | Var | PIP | p-val | OR | CI | Freq | Ref | Alt |
| Class I | Child | CS1 | 1 | rs2428494 | 0.969 | 8.77E−23 | 1.157 | 1.123-1.191 | 0.474 | T | A |
| | Child | CS2 | 1.00 | HLA-C p.11 | 0.573 | 3.12E−19 | 0.806 | 0.768-0.844 | 0.127 | A | S |
| | | | | rs28481932 | 0.426 | 4.36E−19 | 0.807 | 0.769-0.845 | 0.127 | A | G |
| | Adult | CS1 | 1 | rs2428494 | 0.999 | 4.52E−23 | 1.104 | 1.082-1.125 | 0.475 | T | A |
| Class II | Child | CS1 | 1 | rs28407950 | 0.997 | 1.37E−59 | 0.738 | 0.712-0.765 | 0.246 | C | T |
| | Child | CS2 | 0.99 | rs35571244 | 0.495 | 1.07E−17 | 1.253 | 1.189-1.319 | 0.072 | T | C |
| | | | | rs35599935 | 0.201 | 3.39E−17 | 1.251 | 1.187-1.318 | 0.071 | T | C |
| | | | | rs34975158 | 0.171 | 4.73E−17 | 1.249 | 1.185-1.315 | 0.071 | G | A |
| | | | | rs4148878 | 0.056 | 1.60E−16 | 1.243 | 1.180-1.308 | 0.071 | T | G |
| | | | | rs33998906 | 0.029 | 1.42E−15 | 1.242 | 1.177-1.309 | 0.067 | C | T |
| | Adult | CS1 | 0.99 | rs17843580 | 0.078 | 4.01E−49 | 1.167 | 1.143-1.190 | 0.601 | A | G |
| | | | | HLA-DQB1 p.55 | 0.039 | 4.50E−49 | 0.858 | 0.841-0.876 | 0.409 | L, P | R |
| | | | | HLA-DQA1 p.11 | 0.030 | 3.23E−48 | 0.859 | 0.841-0.876 | 0.390 | Y | C |
| | | | | HLA-DQA1 p.18 | 0.030 | 3.23E−48 | 0.859 | 0.841-0.876 | 0.390 | S | F |
| | | | | HLA-DQA1 p.45 | 0.030 | 3.23E−48 | 0.859 | 0.841-0.876 | 0.390 | V | A |
| | | | | HLA-DQA1 p.47 | 0.030 | 3.23E−48 | 0.859 | 0.841-0.876 | 0.390 | C, K, Q | R |
| | | | | HLA-DQA1 p.48 | 0.030 | 3.23E−48 | 0.859 | 0.841-0.876 | 0.390 | L | W |
| | | | | HLA-DQA1 p.50 | 0.030 | 3.23E−48 | 0.859 | 0.841-0.876 | 0.390 | L, V | E |
| | | | | HLA-DQA1 p.52 | 0.030 | 3.23E−48 | 0.859 | 0.841-0.876 | 0.390 | H, R | S |
| | | | | HLA-DQA1 p.53 | 0.030 | 3.23E−48 | 0.859 | 0.841-0.876 | 0.390 | Q, R | K |
| | | | | HLA-DQA1 p.55 | 0.030 | 3.23E−48 | 0.859 | 0.841-0.876 | 0.390 | R | G |
| | | | | HLA-DQA1 p.56 | 0.030 | 3.23E−48 | 0.859 | 0.841-0.876 | 0.390 | R, x | G |
| | | | | HLA-DQA1 p.61 | 0.030 | 3.23E−48 | 0.859 | 0.841-0.876 | 0.390 | F | G |
| | | | | HLA-DQA1 p.64 | 0.030 | 3.23E−48 | 0.859 | 0.841-0.876 | 0.390 | T | R |
| | | | | HLA-DQA1 p.66 | 0.030 | 3.23E−48 | 0.859 | 0.841-0.876 | 0.390 | I | M |
| | | | | HLA-DQA1 p.69 | 0.030 | 3.23E−48 | 0.859 | 0.841-0.876 | 0.390 | L, T | A |
| | | | | HLA-DQA1 p.76 | 0.030 | 3.23E−48 | 0.859 | 0.841-0.876 | 0.390 | L, V | M |
| | | | | HLA-DQA1 p.80 | 0.030 | 3.23E−48 | 0.859 | 0.841-0.876 | 0.390 | S | Y |
| | | | | HLA-DQA1 p.175 | 0.030 | 3.23E−48 | 0.859 | 0.841-0.876 | 0.390 | E, K | Q |
| | | | | HLA-DQA1 p.218 | 0.030 | 3.23E−48 | 0.859 | 0.841-0.876 | 0.390 | R | Q |
| | | | | rs17612788 | 0.023 | 9.67E−49 | 1.165 | 1.141-1.189 | 0.605 | G | A |
| | | | | rs9273084 | 0.015 | 5.25E−48 | 1.164 | 1.140-1.187 | 0.607 | T | C |
| | | | | HLA-DQB1 p.203 | 0.014 | 2.30E−47 | 0.859 | 0.841-0.876 | 0.383 | I, x | V |
| | | | | rs9274660 | 0.011 | 7.88E−48 | 1.163 | 1.139-1.187 | 0.608 | A | G |
| | | | | rs34843907 | 0.010 | 1.51E−47 | 1.163 | 1.139-1.187 | 0.604 | T | G |
| | | | | rs17843619 | 0.010 | 1.11E−47 | 1.163 | 1.139-1.186 | 0.607 | C | A |
| | | | | rs9272629 | 0.009 | 2.34E−46 | 1.161 | 1.137-1.185 | 0.600 | G | T |
| | | | | rs1130034 | 0.009 | 1.27E−47 | 1.163 | 1.139-1.187 | 0.608 | T | C |
| | | | | rs9273497 | 0.009 | 6.01E−47 | 1.163 | 1.139-1.187 | 0.601 | T | C |
| | | | | rs17612576 | 0.009 | 1.16E−47 | 1.163 | 1.139-1.186 | 0.608 | G | A |
| | | | | HLA-DQB1 p.84 | 0.008 | 3.66E−47 | 0.860 | 0.842-0.877 | 0.387 | Q, x | E |
| | | | | HLA-DQB1 p.85 | 0.008 | 3.66E−47 | 0.860 | 0.842-0.877 | 0.387 | L, x | V |
| | | | | HLA-DQB1 p.89 | 0.008 | 3.66E−47 | 0.860 | 0.842-0.877 | 0.387 | T, x | G |
| | | | | HLA-DQB1 p.90 | 0.008 | 3.66E−47 | 0.860 | 0.842-0.877 | 0.387 | T, x | I |
| | | | | HLA-DQB1 p.220 | 0.008 | 3.75E−47 | 0.860 | 0.842-0.877 | 0.387 | H, x | R |
| | | | | HLA-DQB1 p.221 | 0.008 | 3.75E−47 | 0.860 | 0.842-0.877 | 0.387 | H, x | Q |
| | | | | rs9273088 | 0.008 | 1.59E−47 | 1.163 | 1.139-1.186 | 0.608 | A | C |
| | | | | rs3828789 | 0.008 | 1.36E−47 | 1.163 | 1.139-1.186 | 0.608 | G | T |
| | | | | rs1140343 | 0.007 | 6.88E−48 | 1.164 | 1.140-1.188 | 0.603 | T | G |
| | | | | rs1063349 | 0.007 | 1.49E−47 | 1.163 | 1.139-1.186 | 0.608 | T | C |
| | | | | rs17843577 | 0.007 | 3.38E−46 | 1.160 | 1.136-1.184 | 0.600 | C | T |
| | | | | rs9272346 | 0.007 | 1.98E−47 | 1.163 | 1.139-1.186 | 0.608 | G | A |
| | | | | rs9273215 | 0.006 | 1.67E−47 | 1.163 | 1.139-1.186 | 0.608 | G | A |
| | | | | rs9273329 | 0.006 | 1.66E−47 | 1.163 | 1.139-1.186 | 0.608 | G | A |
| | | | | rs9273326 | 0.006 | 1.70E−47 | 1.163 | 1.139-1.186 | 0.608 | T | C |
| | | | | rs17612781 | 0.006 | 2.06E−47 | 1.163 | 1.139-1.186 | 0.608 | T | C |
| | | | | rs9272625 | 0.006 | 3.49E−46 | 1.161 | 1.137-1.185 | 0.599 | T | C |
| | | | | rs17612625 | 0.006 | 1.77E−47 | 1.163 | 1.139-1.186 | 0.608 | A | C |
| | | | | rs17612633 | 0.006 | 1.80E−47 | 1.163 | 1.139-1.186 | 0.608 | G | C |
| | | | | rs17843573 | 0.006 | 1.89E−47 | 1.163 | 1.139-1.186 | 0.608 | C | T |
| | | | | rs9273493 | 0.006 | 1.85E−47 | 1.162 | 1.139-1.186 | 0.608 | T | C |
| | | | | rs1063355 | 0.006 | 1.90E−47 | 1.162 | 1.139-1.186 | 0.608 | T | G |
| | | | | rs9273330 | 0.006 | 3.83E−47 | 1.162 | 1.138-1.186 | 0.605 | C | T |
| | | | | rs17612858 | 0.006 | 1.89E−47 | 1.163 | 1.139-1.186 | 0.608 | A | T |
| | | | | rs9273524 | 0.005 | 2.14E−47 | 1.162 | 1.138-1.186 | 0.608 | T | C |
| | | | | rs9273339 | 0.005 | 2.64E−47 | 1.162 | 1.138-1.186 | 0.608 | G | A |
| | | | | rs17612928 | 0.004 | 9.47E−47 | 1.161 | 1.137-1.185 | 0.602 | A | G |
| | | | | rs1063348 | 0.003 | 2.42E−47 | 1.162 | 1.138-1.186 | 0.605 | A | G |
| | | | | rs17612802 | 0.003 | 8.36E−48 | 1.164 | 1.140-1.188 | 0.602 | C | T |
| | | | | HLA-DQA1 p.-16 | 0.003 | 6.05E−48 | 0.861 | 0.843-0.878 | 0.412 | M | L |
| | Adult | CS2 | 0.96 | rs41269945 | 0.193 | 5.08E−47 | 1.197 | 1.168-1.227 | 0.188 | A | T |
| | | | | rs41269955 | 0.112 | 1.93E−45 | 1.207 | 1.176-1.239 | 0.169 | G | A |
| | | | | HLA-DQA1 p.26 | 0.066 | 3.63E−47 | 1.187 | 1.160-1.215 | 0.204 | T | S |
| | | | | HLA-DQA1 p.47 | 0.066 | 3.63E−47 | 1.187 | 1.160-1.215 | 0.204 | C, K, R | Q |
| | | | | HLA-DQA1 p.56 | 0.066 | 3.63E−47 | 1.187 | 1.160-1.215 | 0.204 | G, x | R |
| | | | | HLA-DQA1 p.76 | 0.066 | 3.63E−47 | 1.187 | 1.160-1.215 | 0.204 | L, M | V |
| | | | | HLA-DQA1 p.187 | 0.066 | 3.63E−47 | 1.187 | 1.160-1.215 | 0.204 | A | T |
| | | | | HLA-DQA1*0301 | 0.057 | 6.91E−47 | 1.187 | 1.159-1.215 | 0.202 | — | — |
| | | | | rs34141382 | 0.056 | 1.48E−45 | 1.200 | 1.170-1.231 | 0.179 | T | C |
| | | | | rs1391371 | 0.051 | 5.75E−47 | 1.187 | 1.159-1.214 | 0.204 | A | T |
| | | | | rs9272461 | 0.028 | 1.84E−46 | 1.187 | 1.159-1.214 | 0.203 | G | A |
| | | | | rs34763586 | 0.016 | 6.01E−45 | 1.194 | 1.165-1.224 | 0.185 | T | C |
| | | | | rs7760841 | 0.014 | 3.69E−44 | 1.196 | 1.166-1.226 | 0.176 | C | T |
| | | | | rs17426593 | 0.013 | 2.67E−45 | 1.191 | 1.162-1.220 | 0.193 | T | C |
| | | | | rs35117964 | 0.008 | 8.13E−43 | 1.187 | 1.158-1.216 | 0.190 | A | G |
| | | | | rs3104413 | 0.007 | 3.07E−44 | 1.184 | 1.156-1.212 | 0.192 | C | G |
| | | | | rs34578704 | 0.007 | 7.07E−43 | 1.204 | 1.172-1.236 | 0.161 | C | A |
| | | | | rs34415150 | 0.006 | 9.06E−44 | 1.188 | 1.159-1.216 | 0.184 | A | G |
| | | | | rs9272785 | 0.005 | 2.92E−44 | 1.189 | 1.160-1.218 | 0.192 | G | A |
| | | | | rs35265698 | 0.005 | 8.55E−44 | 1.184 | 1.155-1.212 | 0.192 | C | G |
| | | | | rs35371668 | 0.005 | 2.51E−43 | 1.190 | 1.160-1.219 | 0.180 | C | T |
| | | | | rs34039593 | 0.005 | 8.50E−44 | 1.184 | 1.155-1.212 | 0.191 | T | G |
| | | | | rs35294087 | 0.004 | 9.99E−44 | 1.183 | 1.155-1.211 | 0.192 | A | G |
| | | | | rs504594 | 0.004 | 8.78E−44 | 1.184 | 1.155-1.212 | 0.191 | C | A |
| | | | | rs9271608 | 0.004 | 8.79E−44 | 1.184 | 1.155-1.212 | 0.191 | A | G |
| | | | | rs3129751 | 0.004 | 9.40E−44 | 1.184 | 1.155-1.212 | 0.191 | A | C |
| | | | | rs35118762 | 0.004 | 1.29E−43 | 1.183 | 1.155-1.211 | 0.192 | C | T |
| | | | | HLA-DRB1 p.-24 | 0.004 | 2.98E−43 | 1.194 | 1.164-1.224 | 0.178 | L, x | F |
| | | | | HLA-DRB1 p.33 | 0.004 | 2.98E−43 | 1.194 | 1.164-1.224 | 0.178 | N | H |
| | | | | HLA-DRB1 p.96 | 0.004 | 2.98E−43 | 1.194 | 1.164-1.224 | 0.178 | E, H, Q, x | Y |
| | | | | HLA-DRB1 p.180 | 0.004 | 2.98E−43 | 1.194 | 1.164-1.224 | 0.178 | V, x | L |
| | | | | HLA-DRB1 p.13 | 0.004 | 3.00E−43 | 1.194 | 1.164-1.224 | 0.178 | F, G | H |
| | | | | rs3997872 | 0.004 | 1.11E−43 | 1.183 | 1.155-1.211 | 0.191 | T | A |

### Fine-Mapping Simulations in the HLA Region

Previous fine-mapping studies excluded the HLA region due to its genomic complexities (Refs. 15-17: incorporated by reference in their entireties). To validate that SuSiE accurately detects multiple independent causal signals in this region, simulations were conducted in each of the HLA class I and class II regions for both COA and AOA, in which 3 causal variants (with non-zero effects) were randomly selected in each of the 4 simulations.

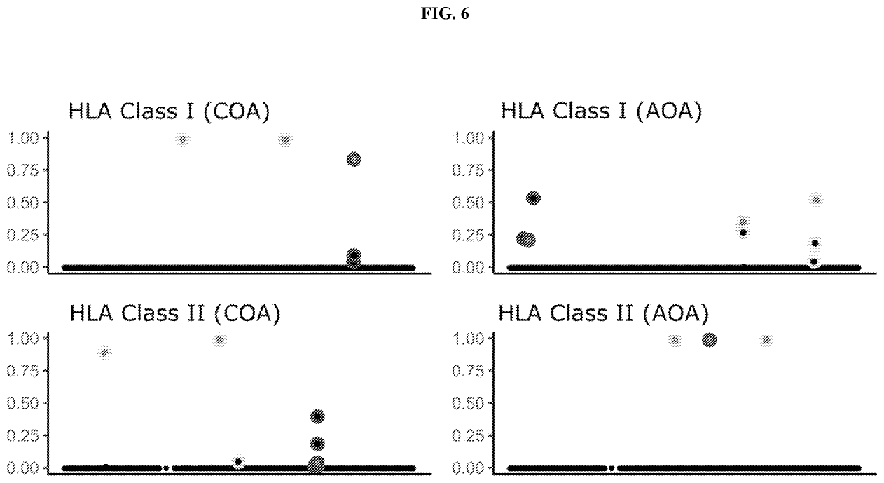

SuSiE correctly detected all 12 causal signals in the class I and class II regions with the designated effect variant within each CS: the designated effect variant had the highest PIP in 10 of the 12 CSs (FIG. 6). These simulations demonstrated that fine-mapping the HLA regions with SuSiE accurately finds CSs containing the true causal variants despite the complexities of this region.

Fine-Mapping eQTLs and Functional Annotations in the HLA Region

Genetic variation can influence disease risk by altering protein function or by impacting expression of disease-associated genes. Studies have demonstrated different functional properties of HLA alleles defined by amino acid polymorphisms and regulatory effects of SNPs on HLA gene expression (Refs. 9, 18-23: incorporated by reference in its entirety). Experiments conducted during development of embodiments herein demonstrated that both types of mechanisms may mediate the effects of HLA genes on asthma risk. The potential regulatory effects of asthma-associated SNPs was assessed using 4 asthma-relevant cell sources of gene expression for which HLA types were known or could be determined. This allowed for mapping of the RNA-seq data against sequences corresponding to each person's known HLA type and the avoidance of mapping biases due to the large number of sequence differences between HLA alleles and reference genome (Ref. 24: incorporated by reference in its entirety). Lymphoblastoid cell lines (LCLs), peripheral blood mononuclear cells (PBMCs), upper airway (nasal) epithelial cells (NECs), and lower airway (bronchial) epithelial cells (BECs) were considered as surrogates for the significant enrichment of genes at COA and AOA GWAS loci in immune, lung and epithelial tissues.

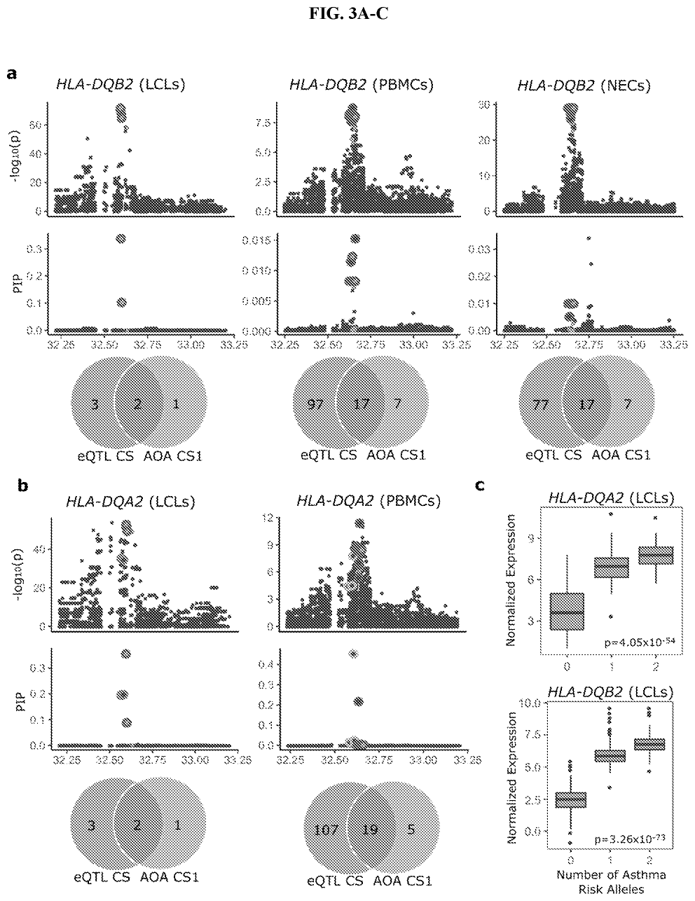

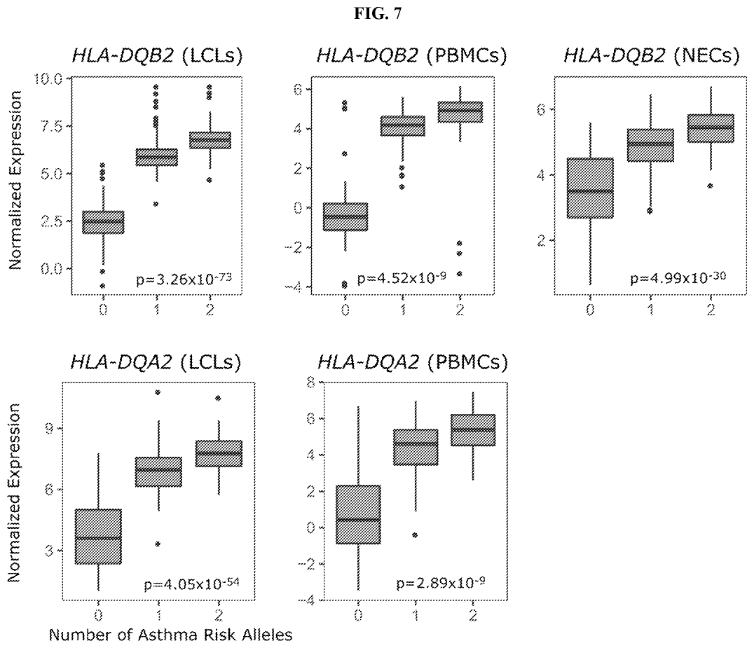

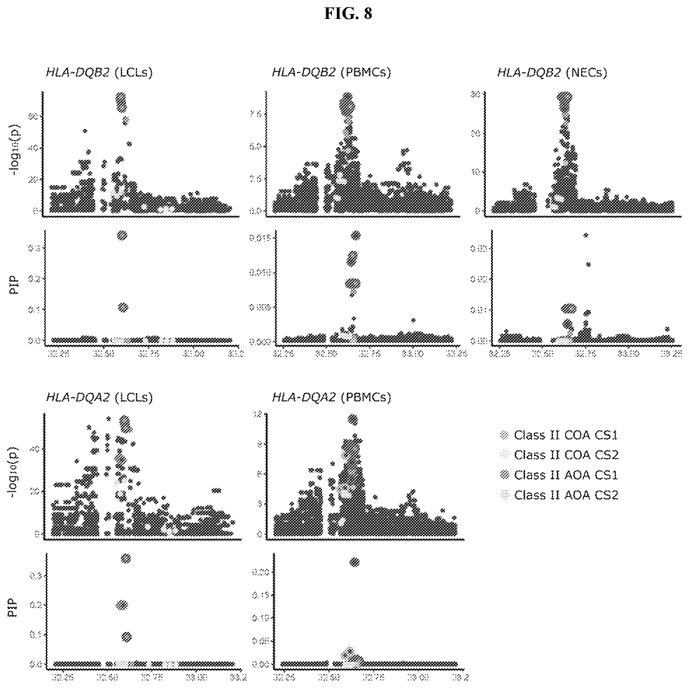

eQTL studies for were performed 148 genes in the class I and class II regions, testing all SNPs±500 kb of the transcription start site of each expressed gene in each cell source. 23 fine-mapping studies were performed using SuSiE, one for each gene with at least one eQTL (FDR<0).05) in that cell type. This included 12 genes in LCLs, 3 in PMBCs, 5 in NECs, and 3 in BECs). Overall, 5 of the fine-mapping studies identified eQTL CSs with SNPs that were also in the class II AOA CS1 (FIG. 3: Table 4). These included CSs with eQTLs for HLA-DQB2 in LCLs, PBMCs, and NECs (FIG. 3a) and HLA-DQA2 in LCLs and PBMCs (FIG. 3b). The AOA risk alleles were associated with increased expression of both HLA-DQA2 and HLA-DQB2 in these cells (FIG. 3c, FIG. 7). None of the SNPs in the eQTL fine-mapping CSs overlapped with class II AOA CS2, with class II COA CS1 or CS2, or with any AOA or COA class I CSs (FIG. 8.).

TABLES 4A-E
SNPs in adult-onset asthma (AOA) class II CS1
that overlap with class II eQTL credible sets.

| | |
|---|---|
| rsid | PIP |

LCL: HLA-DQB2

| | |
|---|---|
| rs9272346 | 0.3411 |
| rs34843907 | 0.1064 |

NEC: HLA-DQB2

| | |
|---|---|
| rs9273084 | 0.010352 |
| rs17843573 | 0.010352 |
| rs17612576 | 0.010352 |
| rs17843577 | 0.010352 |
| rs17843580 | 0.010352 |
| rs17612625 | 0.010352 |
| rs17612633 | 0.010352 |
| rs17612781 | 0.010352 |
| rs17612788 | 0.010352 |
| rs17612802 | 0.010352 |
| rs17612858 | 0.010352 |
| rs9273326 | 0.010352 |
| rs9273329 | 0.010352 |
| rs9273330 | 0.010352 |
| rs9273339 | 0.010352 |
| rs3828789 | 0.010352 |
| rs9274660 | 0.010352 |

LCL: HLA-DQA2

| | |
|---|---|
| rs9272346 | 0.359057 |
| rs34843907 | 0.092248 |

PBMC: HLA-DQA2

| | |
|---|---|
| rs9273339 | 0.0105 |
| rs17612858 | 0.0071 |
| rs9272346 | 0.0062 |
| rs9273084 | 0.0062 |
| rs17843573 | 0.0062 |
| rs17612576 | 0.0062 |
| rs17843577 | 0.0062 |
| rs17843580 | 0.0062 |
| rs17612625 | 0.0062 |
| rs17612633 | 0.0062 |
| rs17612781 | 0.0062 |
| rs17612788 | 0.0062 |
| rs17612802 | 0.0062 |
| rs9273326 | 0.0062 |
| rs9273329 | 0.0062 |
| rs9273330 | 0.0062 |
| rs3828789 | 0.0062 |
| rs9274660 | 0.0062 |
| rs1063355 | 0.0041 |

PBMC: HLA-DQB2

| | |
|---|---|
| rs17612858 | 0.0125 |
| rs9272346 | 0.0084 |
| rs9273084 | 0.0084 |
| rs17843573 | 0.0084 |
| rs17612576 | 0.0084 |
| rs17843577 | 0.0084 |
| rs17843580 | 0.0084 |
| rs17612625 | 0.0084 |
| rs17612633 | 0.0084 |
| rs17612781 | 0.0084 |
| rs17612788 | 0.0084 |
| rs17612802 | 0.0084 |
| rs9273326 | 0.0084 |
| rs9273329 | 0.0084 |
| rs9273330 | 0.0084 |
| rs3828789 | 0.0084 |
| rs9274660 | 0.0084 |

PIP; posterior inclusion probability

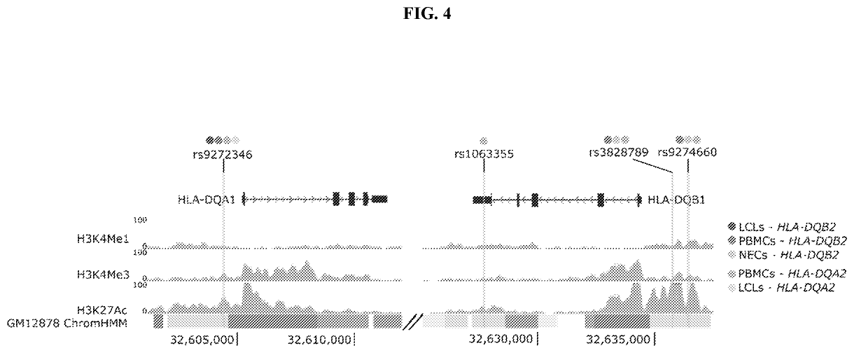

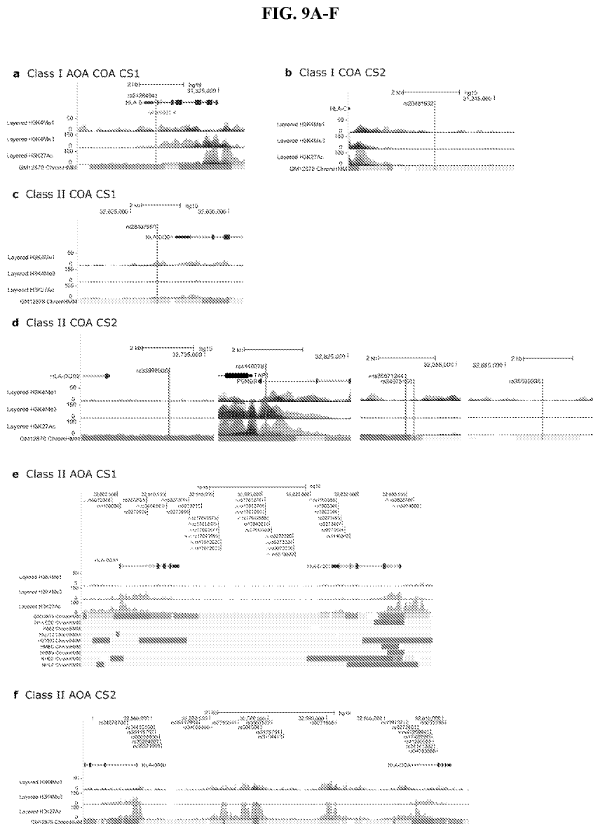

To further prioritize the 20 SNPs that were in both the class II AOA CS1 and the eQTL CSs, these SNPs were overlapped with functional annotations from 9 cell lines (GM12878 (LCLs), H1-hESC, K562, HepG2, HUVEC, HMEC, HSMM, NHEK, NHLF) available from ENCODE (Ref. 25: incorporated by reference in its entirety). Four of the 20 SNPs overlapped an enhancer region only in LCLs (FIG. 4) and resided in or were near (approximately 70 kb to 700 kb) weak enhancer marks in three epithelial cell-derived lines (NHEK, keratinocytes: HMEC, mammary epithelial cells: HEPG2: liver hepatocellular cancer) (FIG. 9). rs9272346, located upstream of HLA-DQA1, was in 4 of 5 of the eQTL CSs. The other 3 SNPs were located near or within HLA-DQB1, with rs1063355, rs3828789, and rs9274660 contained in 1, 3, and 3 of the 5 eQTL CSs, respectively. The AOA GWAS lead SNP (rs17843580), which had the highest PIP in the AOA CS1, and the remaining 15 SNPs did not overlap an enhancer region in any of the cell types (FIG. 9) and are therefore less likely to be causal SNPs at this locus.

eQTL fine-mapping studies of class II genes in 4 asthma-relevant cell types revealed significant effects of SNPs associated with AOA on the expression of HLA-DQB2 and HLA-DQA2 across multiple cell types and overlap with strong enhancer annotations (H3K27ac) in transformed B cells (LCLs). These findings indicate that increased expression of the HLA-DQA2 and HLA-DQB2 genes in peripheral immune cells and increased expression of HLA-DQB2 in airway epithelial cells are associated with AOA risk. Variants in the other COA and AOA CSs did not overlap with any eQTL CSs, indicating distinct gene regulatory mechanisms between COA and AOA in the class II HLA region.

### Structural Visualization of Amino Acid Variants

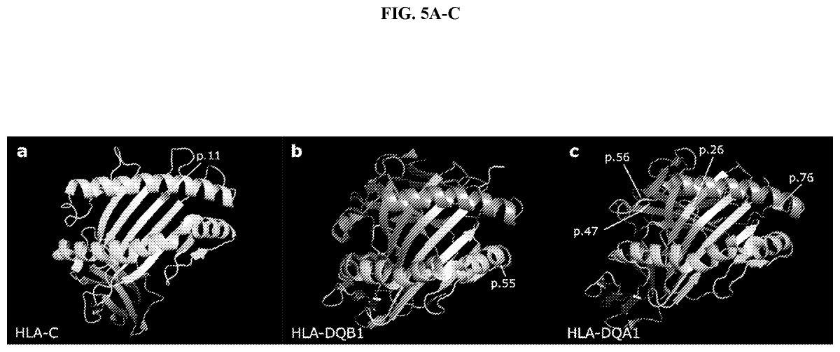

The specific recognition of HLA molecules with bound peptide by the T cell receptor (TCR) drives adaptive immune responses. Experiments were conducted during development of embodiments herein to determine whether the amino acid variants with the highest PIPs in some of the CSs affect peptide presentation or interactions with the TCR. The amino acid in the class I region with the highest PIP in the COA CS2, HLA-C p. 11, is located within the peptide-binding pocket of the HLA-C protein (FIG. 5a). The serine allele was associated with protection from COA and is polar and uncharged: the alanine was associated with risk and is aliphatic and hydrophobic. Thus, both its location and structure could affect peptide presentation and confer functional differences to the HLA-C protein.

The amino acid with the highest PIP in the AOA class II CS1 was at position 55 in the HLA-DQB1 protein (FIG. 5b). Arginine contains a positively charged side chain and was associated with protection from AOA (p=4.50×10⁻⁴⁹: OR=0.86, CI 0.84-0.88). This is a multi-allelic site, with leucine and proline as alternate alleles: both were associated with risk (Leucine: p=3.36×10⁻⁶; OR=1.06, CI 1.03-1.08; Proline: p=1.83×10⁻²⁸: OR=1.12, CI 1.10-1.15). The risk variants were both hydrophobic whereas the protective variant was positively charged and polar. This location may also be in a region that interacts with the TCR. Because this variant is in strong LD with other eQTLs in the class II AOA CS1 (median r²=0.99), it is unclear if this variant, the eQTLs, or both are causal for AOA.

Among the 10 amino acids in class II AOA CS2, HLA-DQA1 Ser26, Gln47, Arg56, and Val76 were in perfect LD with each other, had the highest PIPs of the amino acid polymorphisms in CS2, and were associated with AOA risk. These amino acids are present exclusively on the HLA-DQA1*03:01, 03:02, and 03:03 alleles. Of these 4 amino acids, Ser26 is in the peptide-binding pocket and Val76 is in a region that may interact with the TCR (FIG. 5c). The other amino acid polymorphisms were not in regions with obvious functional effects. At position 26, both the risk-associated serine and the protection-associated threonine have polar uncharged side chains, although the serine sidechain is smaller. Position 76 was multiallelic, with all 3 amino acids (valine, leucine, and methionine) having hydrophobic side chains, and valine having the smallest molecular weight. Position 187 was also perfectly correlated with these amino acids and captured in CS2 but was not part of the crystal structure. These data indicate that HLA-DQA1*03 alleles are risk alleles for AOA.

These data indicate that HLA class II alleles and/or amino acid polymorphisms contribute to asthma. Experiments conducted during development of embodiments herein demonstrate a role for HLA-DQA1 and HLA-DQB1 proteins in risk for AOA and the HLA-C protein in risk for COA.

### Summary of Experimental Results

The HLA region is associated with more diseases than any other region of the genome (Ref. 26: incorporated by reference in its entirety), and variation in this region has been strongly and consistently associated with asthma risk in GWASs (Refs. 2-5 and 27-29; incorporated by reference in their entireties), but the causal variants and genes have been unknown. Most previous large studies focused only on SNPs that do not fully capture the extensive protein polymorphisms at this locus. A recent study reported colocalizations between eQTLs for HLA-B, HLA-DQB1, HLA-DQA1, HLA-DRA, TAP1, and RNF5 in induced pluripotent stem cells with asthma GWAS SNPs (Ref. 22: incorporated by reference in its entirety). However, they did not separate COA and AOA, include HLA-DQA2 or HLA-DQB2 expression, examine HLA allele or amino acid associations, or study asthma-relevant cell types, as was done in the fine-mapping study conducted during development of embodiments herein. Causal variation was prioritized by examining functional annotations from ENCODE. In the class I region, both a COA-specific association that may be mediated by HLA-C protein coding variation and a shared causal variant with unknown function were identified. The study did not find any shared causal variation between COA and AOA in the class II region. The data strongly indicates that AOA is mediated, at least in part, by differential expression of the nonclassical HLA-DQA2/HLA-DQB2 genes and protein coding variation associated with the HLA-DQA1*03 alleles.

Fine-mapping studies revealed a lead GWAS SNP in an intron of HLA-B in the class I region that was putatively causal for both COA and AOA (rs2428494). However, rs2428494 was not in any eQTL CSs and is therefore not likely conferring asthma risk by modifying expression of genes in our study. This SNP was not an eQTL for any genes in GTEx (Ref. 30); incorporated by reference in its entirety) and did not reside in ENCODE (ref. 25: incorporated by reference in its entirety) cis regulatory elements (FIG. 9). It was the lead class I region SNP in a meta-analysis of GWASs for allergic rhinitis, a common co-morbidity with asthma, with the same allele associated with risk (Ref. 31: incorporated by reference in its entirety).

A second CS in the class I region contained a SNP (rs28481932) and an HLA-C amino acid polymorphism at position 11 and was specific to COA. Based on gene expression and co-localizations with ENCODE annotations (FIG. 9), there was not functional evidence for the SNP to mediate its effects through gene expression. However, the HLA-C amino acid polymorphism had a higher PIP and its location in the HLA-C protein indicates function. Along with other class I classical HLA genes, HLA-C is expressed on the cell surface of nearly all cells and can present intracellular peptides to cytotoxic CD8+ T cells, which can trigger an immune response. Position 11 lies in a beta sheet in the peptide-binding pocket and the amino acid substitution may change the peptide binding properties and impact antigen recognition. HLA-C is also a ligand for killer immunoglobulin receptors (KIRs), which are expressed on natural killer (NK) cells (Ref. 33: incorporated by reference in its entirety), and changes in peptide presentation can also impact NK cell recognition (ref. 34: incorporated by reference in its entirety), which may present another potential role for this variant in COA (Ref. 35-36; incorporated by reference in their entireties).

In the class II region. 2 CSs were found for COA, neither of which overlapped with the class II AOA CSs. One contained a single SNP (rs28407950). Despite being predicted to reside in an active enhancer in LCLs (FIG. 9), eQTL fine-mapping indicates that this SNP was not the likely causal variant for expression of any genes in our study. None of the class II COA CS2 SNPs were in the eQTL CSs and therefore not likely causal for gene expression. The fact that no HLA alleles or amino acids were included in either COA CS largely rules out protein variation mediating risk at this locus for COA.

Fine-mapping studies identified the same causal variation underlying both AOA risk and expression of HLA-DQA2/DQB2 genes at the class II region. Although previous studies have interpreted GWAS results to implicate the highly polymorphic, classical HLA-DQA1 and HLA-DQB1 class II genes in asthma risk (Refs. 27, 29, 37: incorporated by reference in their entireties), experiments conducted during development of embodiments herein revealed strong associations with the non-polymorphic, non-classical HLA-DQA2 and HLA-DQB2 genes in risk for AOA. The HLA-DQA2/HLA-DQB2 genes are paralogous to HLA-DQA1/DQB1 and highly conserved (Refs. 38.39; incorporated by reference in their entireties), although the former has few amino acid polymorphisms in contrast to the highly polymorphic coding regions of the latter. Little is known about the functions of HLA-DQA2 and HLA-DQB2, but they were shown to form heterodimers and participate in antigen presentation on Langerhans cells (Ref. 40; incorporated by reference in its entirety). SNPs in the AOA CS1 were associated with increased HLA-DQB2 and HLA-DQA2 expression in the different cell types, pointing to their potentially broad effects in both immune and epithelial tissues. The finding that asthma-associated SNPs in the class II region were associated with increased HLA-DQA2/DQB2 expression is consistent with results showing that increased expression of HLA-DQA2/DQB2 predicted from GTEx data were among the strongest gene-based associations with asthma risk (Refs. 3, 41: incorporated by reference in their entireties). Experiments conducted during development of embodiments herein strongly implicate increased expression of the highly conserved yet under-characterized HLA-DQA2/DQB2 genes as potential mediators of risk for AOA.

Experiments conducted during development of embodiments herein also indicate that HLA-DQA1*03 alleles also play an important role in AOA risk. The class II AOA CS2 contained an HLA-DQA1*03:01 allele and 5 amino acids that are on the common HLA-DQA1*03 alleles in subjects: some of these polymorphisms are in regions with potential impact on peptide presentation and TCR interactions (FIG. 5c). HLA-DQA1*03:01 may therefore be the causal variant for AOA in CS2. T cell activation and proliferation is driven by both differential expression levels and protein coding variation in the HLA genes which may affect binding affinities to different peptides (Refs. 42-43: incorporated by reference in their entireties). The class II region was the most significantly associated locus for AOA, indicating a particularly important role for immune genes in AOA. Experiments conducted during development of embodiments herein indicate that both increased expression of the HLA-DQA2/HLA-DQB2 genes and coding variation in the HLA-DQA1*03 protein mediate risk at the most significant AOA GWAS locus.

Experiments conducted during development of embodiments herein highlight a role for both gene expression levels and protein coding changes in the HLA-DQ genes in AOA and for HLA-C protein coding variation in COA. In addition to age of asthma onset, many other known epidemiological factors distinguish asthma with onset in childhood compared to onset after puberty, including sex-specific prevalence, the importance of respiratory viral infections, and comorbidities with allergic diseases or obesity, as examples (Ref. 49; incorporated by reference in its entirety).

### Methods

### Study Subjects

COA and AOA HLA loci were examined in the same adult individuals from the UKB and using the same inclusion/exclusion criteria and phenotype definitions reported in Pividori, et al. (Ref. 3: incorporated by reference in its entirety). COA was defined as onset younger than 12 years old (n=9,432), AOA as onset between 26-65 years old (n=21,556), and controls as having no reported asthma at the latest age of study (n=318,167).

### Genotypes and HLA Alleles

Allele dosages were extracted for n=349,155 individuals from genotyped and imputed SNPs from UKB v3 within the boundaries of the asthma HLA loci in the COA and AOA GWAS as defined by Pividori et al., using the rbgen 0.1 package in R 3.6.1. All SNPs that passed the following genotype quality control filters were included: call rate>95% or information score>80%, Hardy-Weinberg equilibrium test p-value>1×10⁻¹⁰, and minor allele frequency (MAF)>0.1%, as previously described³. In summary, for the analyses, 8,624 SNPs the HLA class I locus and 10,006 SNPs at the class II locus were used.

Four-digit resolution of classical HLA allele dosages, imputed from SNP data, were available from the UK Biobank (Ref. 7: incorporated by reference in its entirety). After filtering out low-frequency HLA variants (<1%), there were a total of 78 alleles for the HLA-A (n=13), HLA-B (n=18), HLA-C (n=14), HLA-DRB1 (n=15), HLA-DQB1 (n=12), and HLA-DQA1 (n=7) genes.

The imputed HLA allele dosages were translated to their corresponding amino acid dosages using publicly available data from SNP2HLA (ref. 50; incorporated by reference in its entirety) (http://software.broadinstitute.org/mpg/snp2hla/) that map HLA alleles to their amino acid sequences. Amino acid polymorphisms were coded as biallelic. Multiallelic positions were coded with a binary marker that corresponded to the absence or presence of that amino acid. After filtering out low frequency amino acid polymorphisms (<1%) there were 263 biallelic and 257 multiallelic amino acid sites for a total of 741 total amino acids tested.

Base-pair positions for variants, genes and other genomic features are based on Human Genome Assembly hg19.

### HLA Allele and Amino Acid Associations

Logistic regression was performed to test for associations between each of the imputed HLA alleles with COA and AOA. Sex and the first 10 ancestral principal components (PCs) were included as covariates, as was done in the GWASs. Associations with each amino acid polymorphisms with frequencies≥0.01 were tested. Variants that were considered shared between COA and AOA were genome-wide significant for COA and/or AOA, had p<0.05 for both, and overlapping 95% Cis.

### Fine-Mapping the HLA Region

Sum of Single Effects (SuSiE)(Ref. 14: incorporated by reference in its entirety) (susieR R package version 0.9.0) was used to fine-map the asthma-associated HLA loci and determine putatively causal variants for COA and AOA, separately in the class I and class II regions. To assess whether SNPs, amino acid polymorphisms, and/or HLA alleles were causal for asthma risk, genotype dosages for these 3 types of polymorphisms were included in a genotype matrix: class I SNPs, HLA alleles, and HLA amino acid polymorphisms were considered together for the fine-mapping studies in the class I region: class II SNPs, HLA alleles, and HLA amino acid polymorphisms were examined together for the class II region.

The susieR R package does not currently allow for the inclusion of covariates, so sex and the first 10 ancestral PCs were regressed out of the genotype matrix and phenotype vector using linear regression: the residuals of the genotype matrix and phenotype vector were used as inputs to SuSIE. Although the SuSiE method is based on a linear regression, and therefore is best suited for quantitative traits, and not a binary (case-control) outcome, the linear-regression-based likelihood provides a good approximation to a logistic-regression-based likelihood because the estimates ORs are all <1.3, the allele frequencies are not too extreme and the sample size (here, limited by the smaller number of cases) is large (Refs. 44-46: incorporated by reference in their entireties). It was assumed at most L=10 causal variants and set susieR to estimate the residual and prior variances. Only level-95% CSs (coverage=0.95) was retained. The additional step of discarding CSs in which the “purity” (smallest absolute correlation among all pairs of variants within the CS) was less than 0.75 was taken. Additionally, one CS was removed due to the variants having high p-values (p>0.001) to ensure we were excluding any possible artifacts.

### HLA Fine-Mapping Simulations

Because the HLA region is extraordinarily complex, the performance of SuSiE in this region was assessed by simulation. Case/control status was simulated using existing genotype and covariate data to leverage the true correlation structure in the class I and class II regions. Using the genotype matrix X and covariate matrix Z, the individual-level log-odds of asthma was set to be

$$ln \frac{{p}_{i}}{1 - {p}_{i}} = {\sum }_{j} {β}_{j} {X}_{i j} + {\sum }_{k} {δ}_{k} {Z}_{i k} + μ$$

for individual i, SNPs j, covariates k, fixed effect vectors β and δ, and a fixed intercept μ. The true matrix of covariate and covariate effects δ estimated from a logistic regression was used, separately for COA and AOA simulations. βⱼ was set to 0 for all non-causal variants, and the βⱼ for the causal variants in the class I and class II regions were set using effect sizes similar to what was found in the Pividori GWASs (Ref. 3: incorporated by reference in its entirety).

Four simulations were conducted by randomly selecting 3 variants in the class I and class II regions for COA and AOA, for a total of 12 causal variants. Case-control status was then simulated for each individual as Yᵢ˜Bernoulli(pᵢ) independently and regressed out the covariates in Z from X and Y. SuSiE was then used to test how well the method recovers the causal effects over each simulation.

Gene Expression and eQTL Studies in Lymphoblastoid Cell Line (LCLs)

To test whether SNPs from the fine-mapping results using SuSiE are eQTLs in asthma-relevant cell types, RNA-seq data previously collected from LCLs from 398 Hutterites was examined (Ref. 51: incorporated by reference in its entirety). The Hutterites are a founder population of European descent with well characterized HLA types for the polymorphic HLA-A, HLA-B, HLA-C, HLA-E, HLA-G, HLA-DPB1, HLA-DRB1, HLA-DQB1, and HLA-DQA1 genes (Ref. 52: incorporated by reference in its entirety). The sample was composed of 191 males and 207 females who were between the ages of 10 and 60 at the time of sample collection. Informed consent was obtained from all participants under University of Chicago IRB-approved protocols.

Standard RNA-seq pipelines that map reads to a reference genome can provide biased expression estimates at the highly polymorphic HLA loci due to the potentially large number of differences between the sequence of an individual's HLA type and the reference sequence used for mapping. Expression estimates can be improved by mapping RNA-seq reads to the sequences for each individual's known HLA type (Ref. 24: incorporated by reference in its entirety). For the polymorphic HLA genes, RNA-seq reads were aligned to reference sequences from the IMGT database (Ref. 53: incorporated by reference in its entirety) for each individual's known HLA type, removing duplicate reads with wasp (Ref. 54; incorporated by reference in its entirety). Sequencing reads were mapped and quantified using STAR/2.6.1 (Ref. 55: incorporated by reference in its entirety) for other genes. Samples with >7M uniquely mapped reads underwent trimmed means of M-value (TMM) normalization and voom transformation (ref. 56; incorporated by reference in its entirety). Extraction date and sequencing batch were corrected for with limma (ref. 57: incorporated by reference in its entirety).

To perform eQTL mapping, associations between SNPs and expression of genes in the HLA class I and class II regions were performed with Genome-wide Efficient Mixed Model Association (GEMMA) (Ref. 58: incorporated by reference in its entirety) using a kinship matrix to correct for relatedness between Hutterite individuals. A linear mixed model (LMM) was used, including age and sex as covariates and considered all variants within 0.5 Mb of the transcription start site (TSS) of each expressed gene.

Gene Expression and eQTL Studies in Peripheral Blood Mononuclear Cells (PBMCs)

Unstimulated PBMC RNAseq data was examined from n=133 (80 males, 53 females) African-American children from the URban Environment and Childhood Asthma (URECA) birth cohort who were 2 years old at the time of sample collection (Refs. 59-60; incorporated by reference in their entireties). HLA-LA (Ref. 61: incorporated by reference in its entirety) was used to infer HLA types from whole-genome sequences for HLA-A, HLA-B, HLA-C, HLA-E, HLA-F, HLA-DRB1, HLA-DQB1, HLA-DQA1, HLA-DPB1, and HLA-DPA1. Reads were mapped and normalized as described. To perform eQTL mapping, linear regressions were examined with QTLtools (ref. 62: incorporated by reference in its entirety), using a nominal pass and cis-window size of 0.5 Mb around the TSS. Sex, reported ancestry, collection site, the top 3 ancestral PCs, and 19 latent factors were included as covariates in the analysis.

Gene Expression and eQTL Studies in Nasal Epithelial Cells (NECs)

Nasal NEC RNAseq data was examined from 189 (92 females, 97 males) African-American children (age 11 at time of sample collection) from the URECA cohort (ref. 63; incorporated by reference in its entirety). As described above for the PBMCs, HLA-LA was used to infer HLA types from whole-genome sequences, mapped reads as described above, and used QTLtools to perform eQTL mapping, using sex, the top 3 ancestral PCs, collection site, epithelial cell proportion, sequencing batch, and 7 latent factors as covariates in the analysis.

Gene Expression and eQTL Studies in Bronchial Epithelial Cells (BECs)

BEC RNAseq data from adults who participated in previous studies were examined (Ref. 64: incorporated by reference in its entirety). This study focused on data for 45 European American adults (33 asthma cases, 12 controls: 11 males, 34 females) between the ages of 19-60 years at the time of sample collection. Informed consent was obtained from all participants under University of Chicago IRB-approved protocols.

HLA types were imputed for HLA-A, HLA-B, HLA-C, HLA-DRB1, HLA-DQB1, and HLA-DQA1 in the European-American individuals using SNP2HLA (Ref. 50; incorporated by reference in its entirety) from genotype data using the Type 1 Diabetes Consortium (T1DGC) HLA reference panel (Ref. 65: incorporated by reference in its entirety). Reads were mapped and normalized. Sequencing batch was corrected for with limma.

In order to determine if any variants were associated with expression of genes in the HLA region, linear regressions was performed between genotypes and gene expression with QTLtools using a cis-window size of 0.5 Mb around the TSS using a nominal pass and correcting for sex, age, and smoking status.

Fine-Mapping eQTLs

Fine-mapping was performed in the LCLs, PBMCs, NECs, and BECs using SuSiE for the expression of genes in which SNPs in any of the COA or AOA CSs were significantly associated with their expression at an FDR threshold of 5%. This included 14 genes: MIR6891, PSBM9, TAP1, TAP2, PPT2, HLA-B, HLA-DQA1, HLA-DQB1, HLA-DQA2, HLA-DQB2, HLA-DRB5, HLA-DRB6, HLA-DRB9, and HLA-DPB2. Overall, we performed 23 fine-mapping studies: 12 genes in LCLs (MIR6891, PSMB9, TAP1, TAP2, PPT2, HLA-B, HLA-DQA2, HLA-DQB2, HLA-DRB5, HLA-DRB6, HLA-DRB9, HLA-DPB2), 3 genes in PBMCs (HLA-DQA2, HLA-DQB2, HLA-DRB6), 5 genes in NECs (HLA-DQA1, HLA-DQB1, HLA-DQA2, HLA-DQB2, HLA-DRB6), and 3 genes in BECs (HLA-DQA2, HLA-DQB2, HLA-DRB6).

In each of the datasets, the same covariates used in each of the eQTL studies were regressed out. Because SuSiE is unable to account for missing data, individuals that were missing genotypes for any of the SNPs in the COA or AOA CSs were filtered out, and then other SNPs with any missing genotypes were filtered out.

SuSiE was then used to fine-map eQTLs using a window of +0.5 Mb around the TSS of each gene. SNPs in any of the eQTL CSs were then compared to the SNPs found in the COA and AOA GWAS CSs to assess whether there was overlap.

### Structural Visualization of Amino Acid Variants

Based on results of fine-mapping, visualization of amino acid polymorphisms was performed for HLA-C p.11, HLA-DQB1 p.55, and HLA-DQA1 p.26, p.47, p.56, and p.76. The amino acid polymorphisms were aligned to their positions on the protein. Crystal X-ray structures from the Protein Data Bank (PDB) were obtained for HLA molecules containing the risk/protective AA variants of interest if possible: 5VGE (HLA-C*07:02), 6DIG (HLA-DQA1*01:02/HLA-DQB1*06:02), and 4D8P (HLA-DQA1*03:01/HLA-DQB1*02:01) and visualized them with PyMOL v2.0.7 (pymol.org/2/) (Refs. 67-69: incorporated by reference in their entireties). The NCBI Amino Acid Explorer was used to assess differences in amino acids.

### Conditional Analyses to Assess Independent Effects

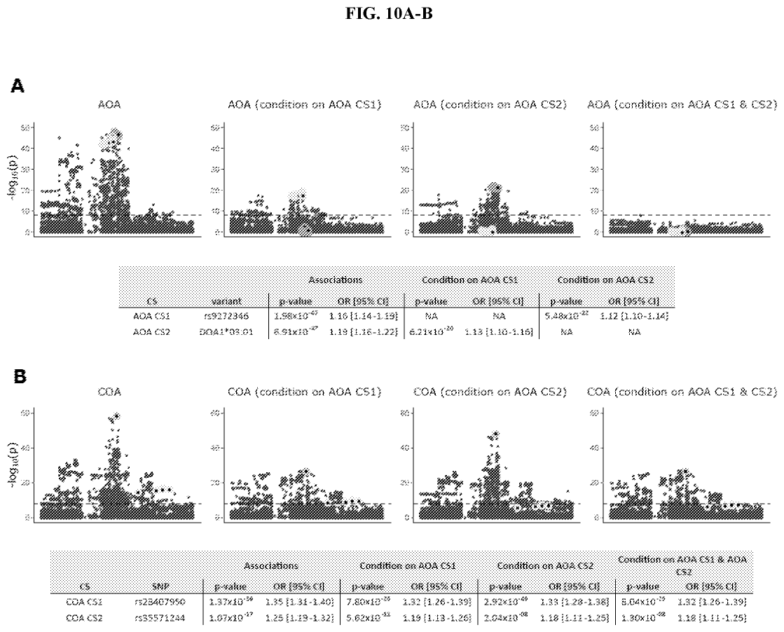

To determine whether the candidate eQTL SNP for HLA-DQA2 and HLA-DQB2 (rs9272346) in CS1 and the HLA-DQA1*03:01 allele in CS2 accounts for all the association signal at the class II AOA locus, three conditional analyses were performed in which the number of alleles for rs9272346, for HLA-DQA1*03:01, and for both were included as covariates in association tests of variants at this locus with AOA (FIG. 10A). In the first conditional analysis including number of rs9272346 (CS1) alleles as a covariate, the AOA association signal for HLA-DQA1*03:01 remained genome-wide significant: in the second conditional analysis including the number of HLA-DQA1*03:01 alleles (CS2) as a covariate, the AOA association signal for rs9272346 also remained genome-wide significant. When both rs9272346 and HLA-DQA1*03:01 were included as covariates, the significance across the locus was reduced. These analyses indicate that the most significant asthma locus in AOA is due to variation in two credible sets, whose effects are likely attributable to their impact on expression of the HLA-DQA2 and HLA-DQB2 genes and of the HLA-DQA1*03:01 allele.

To confirm that the two class II AOA putatively causal variants do not contribute to class II COA risk, the analysis above was repeated for SNPs at the COA class II GWAS locus (FIG. 10B). When conditioning on either the AOA CS1 SNP rs9272346, the AOA CS2 HLA-DQA1*03:01 allele, or both, the COA associations remain genome-wide significant, although the magnitudes of the associations are reduced, likely due to including additional covariates in the model and the LD in the region. These results further indicate that risk for COA and AOA are due to different causal variants in the HLA class II region.

Conditional analyses were performed to assess the independence between the class I and class II signals. For each of the COA class I variants, association with COA (and AOA) was tested after conditioning on the tag class II SNPs and vice versa for the class II region. For all results, the odds ratios (ORs) are largely similar and the 95% confidence intervals (CIs) overlap between the marginal and conditional associations, indicating that the class I and class II signals are indeed independent.

### Replication of Fine-Mapping Results

To replicate the COA and AOA putatively causal SNPs identified in the UK Biobank White British ancestry individuals, a replication cohort of UK Biobank multi-ethnic individuals who were initially excluded was used. Asthma and allergy phenotypes were defined using the same criteria as in the discovery sample. The prevalence of both were similar in the discovery and replication samples. To allow for allele frequency and effect size heterogeneity between the replication cohorts (n=43,449 White British, n=10,327 Asian or Asian British, n=7,637 Black or Black British), each variant was tested for association with COA and AOA within each cohort and then performed a meta-analysis of the results. It was required that the same allele is associated with asthma with the same direction of effect as in the discovery cohort. All of the variants, except the class II COA CS2 and the class I AOA CS1 SNPs, replicated at a significance threshold adjusted for multiple testing of 5.0×10⁻³ (Table 5). Additionally, the HLA-DQA1*03:01 allele had the most significant association for AOA compared to the other HLA alleles tested.

TABLE 5
Results of Replication Meta-analysis.

| | | | Allele | | | |
|---|---|---|---|---|---|---|
| Group | CS | Variant | (non-risk/risk) | p-value | OR | 95% CI |
| COA | Class I CS1 | rs2428494 | T/A | 9.72 × 10⁻⁰⁴ | 1.13 | 1.05-1.21 |
| | Class I CS2 | HLA-C p.11 | Ser/Ala | 2.66 × 10⁻⁰³ | 1.16 | 1.05-1.29 |
| | Class II CS1 | rs28407950 | T/C | 1.30 × 10⁻⁰⁹ | 1.29 | 1.19-1.40 |
| | Class II CS2 | rs35571244 | T/C | 2.74 × 10⁻⁰¹ | 1.08 | 0.94-1.25 |
| AOA | Class I CS1 | rs2428494 | T/A | 1.27 × 10⁻⁰² | 1.06 | 1.01-1.12 |
| | Class II CS1 | rs9274660 | A/G | 6.10 × 10⁻⁰⁸ | 1.14 | 1.09-1.20 |
| | | rs3828789 | G/T | 4.59 × 10⁻⁰⁸ | 1.15 | 1.09-1.20 |
| | | rs1063355 | T/G | 4.31 × 10⁻⁰⁸ | 1.15 | 1.09-1.20 |
| | | rs9272346 | G/A | 7.52 × 10⁻⁰⁸ | 1.14 | 1.09-1.20 |
| | Class II CS2 | HLA-DQAI | *03:01 | 1.27 × 10⁻⁰⁴ | 1.13 | 1.06-1.21 |

P-values, odds ratios (ORs), and 95% confidence intervals (95% CI) in the replication cohorts are reported for each of the candidate variants from the discovery COA and AOA credible sets.

All but two of the candidate variants (AOA rs2428494 and COA rs35571244) from the discovery cohort were significantly associated with COA or AOA in the replication cohort with the same bn6mdirection of effect. Both variants were nominally associated with COA or AOA, with the same directions of effect, but were not significant after multiple test correction.

HLA-DQ2 Expression in Lung scRNAseq

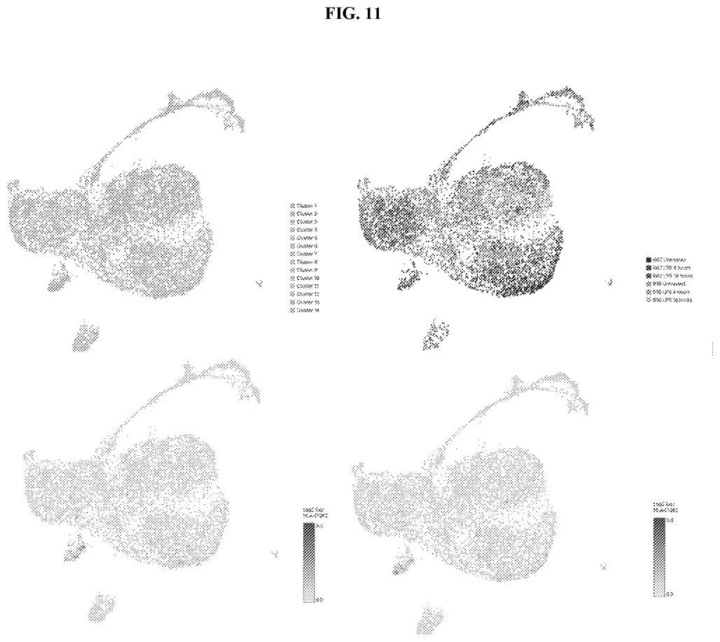

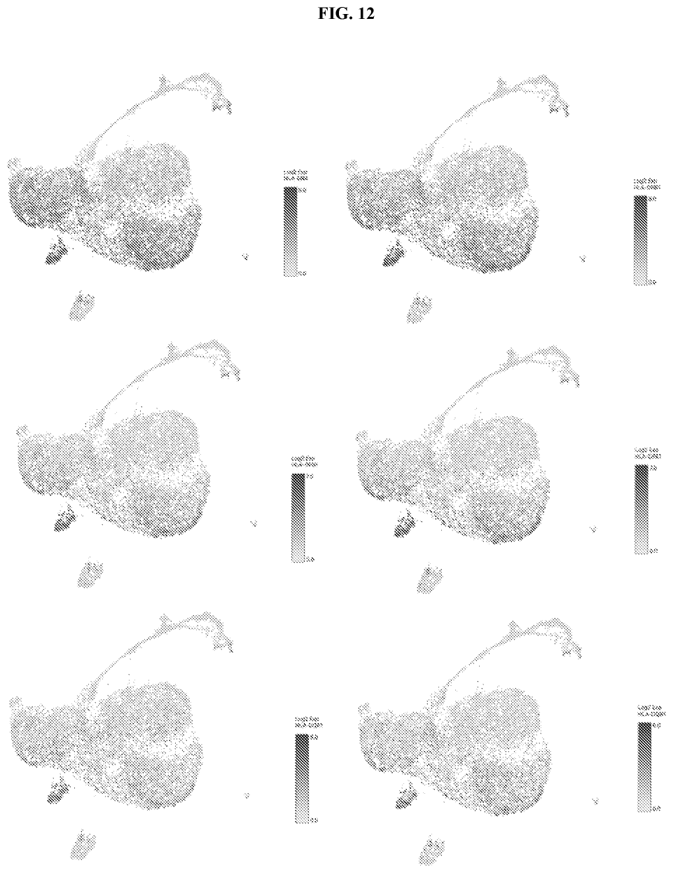

Using a single cell RNA sequencing (scRNAseq) approach, the expression of HLA-DQA2 and HLA-DQB2 has been identified in a subset of lung immune cells (FIG. 11). These genes were more highly expressed and expressed more frequently in a cluster of cells (Cluster 13) that were distinct from macrophage cells (Clusters1-7) as well as B and T cells (Cluster 11). Cluster 13 was also characterized by very high expression of all the classical HLA class 2 genes (FIG. 12). Because the hallmark of dendritic cells and dendritic-like cells, such as Langerhans cells, is very high expression of HLA class 2 genes, this observation indicates that cells expressing HLA-DQA2 and/or HLA-DQB2 are likely to be dendritic cells or dendritic-like cells, such as Langerhans cells.

Differential gene expression analysis of cells in Cluster 13 that expressed HLA-DQA2 and/or HLA-DQB2 (HLA-DQ2 cells) versus all other cells in the experiment identified 76 genes with increased expression (Benjamini-Hochberg adjusted p-value<0.05; Table 6). This included multiple MHC class 2 genes, providing objective evidence for increased expression of these genes in HLA-DQ2 cells and indicating that these cells are active in antigen presentation.

TABLE 6
Genes with increased expression in HLA-DQ2+ cells.

| | | Average | Log₂ fold | |
|---|---|---|---|---|
| | | log₂ | increase | |
| | | expression | compared | |
| | Feature | in HLA- | with other | |
| Feature ID | name | DQ2 cells | cells | P-value |
| ENSG00000166676 | TVP23A | 3.32 | 6.40 | 1.83E−12 |
| ENSG00000241106 | HLA-DOB | 1.29 | 6.17 | 1.36E−10 |
| ENSG00000075618 | FSCN1 | 18.25 | 5.92 | 1.46E−10 |
| ENSG00000122025 | FLT3 | 3.10 | 5.77 | 2.06E−09 |
| ENSG00000136111 | TBC1D4 | 7.70 | 5.62 | 5.79E−09 |
| ENSG00000102970 | CCL17 | 19.72 | 6.48 | 2.47E−08 |
| ENSG00000102962 | CCL22 | 66.78 | 5.63 | 5.62E−08 |
| ENSG00000137571 | SLCO5A1 | 3.85 | 5.52 | 6.59E−08 |
| ENSG00000126353 | CCR7 | 25.48 | 5.36 | 9.57E−08 |
| ENSG00000078081 | LAMP3 | 14.73 | 5.27 | 1.96E−07 |
| ENSG00000132329 | RAMP1 | 10.51 | 5.37 | 2.06E−07 |
| ENSG00000120658 | ENOX1 | 2.87 | 5.48 | 2.55E−07 |
| ENSG00000078589 | P2RY10 | 1.17 | 5.35 | 2.70E−07 |
| ENSG00000221887 | HMSD | 1.36 | 5.51 | 3.01E−07 |
| ENSG00000179593 | ALOX15B | 1.04 | 5.50 | 6.10E−07 |
| ENSG00000229425 | AJ009632.2 | 1.27 | 5.80 | 7.12E−07 |
| ENSG00000064989 | CALCRL | 3.63 | 5.45 | 7.71E−07 |
| ENSG00000077984 | CST7 | 11.36 | 5.16 | 1.46E−06 |
| ENSG00000090104 | RGS1 | 3.29 | 5.14 | 2.16E−06 |
| ENSG00000105246 | EBI3 | 8.34 | 4.97 | 2.79E−06 |
| ENSG00000213145 | CRIP1 | 50.54 | 4.98 | 5.18E−06 |
| ENSG00000054219 | LY75 | 3.76 | 4.93 | 5.20E−06 |
| ENSG00000205755 | CRLF2 | 4.50 | 4.82 | 1.50E−05 |
| ENSG00000137265 | IRF4 | 3.45 | 4.84 | 1.69E−05 |
| ENSG00000135373 | EHF | 3.02 | 4.76 | 2.33E−05 |
| ENSG00000120278 | PLEKHG1 | 1.79 | 5.05 | 2.63E−05 |
| ENSG00000101445 | PPP1R16B | 2.52 | 4.86 | 2.63E−05 |
| ENSG00000179399 | GPC5 | 2.06 | 5.22 | 4.45E−05 |
| ENSG00000182732 | RGS6 | 2.27 | 5.33 | 6.65E−05 |
| ENSG00000164236 | ANKRD33B | 4.46 | 4.45 | 2.21E−04 |
| ENSG00000135074 | ADAM19 | 8.40 | 4.49 | 2.88E−04 |
| ENSG00000070190 | DAPP1 | 7.55 | 4.34 | 3.45E−04 |
| ENSG00000251301 | LINC02384 | 1.18 | 4.58 | 4.11E−04 |
| ENSG00000179862 | CITED4 | 1.73 | 4.52 | 4.21E−04 |
| ENSG00000120833 | SOCS2 | 2.20 | 4.36 | 4.52E−04 |
| ENSG00000064042 | LIMCH1 | 1.63 | 4.63 | 4.96E−04 |
| ENSG00000163389 | POGLUT1 | 2.30 | 4.28 | 7.21E−04 |
| ENSG00000133401 | PDZD2 | 1.10 | 4.55 | 9.61E−04 |
| ENSG00000047365 | ARAP2 | 8.27 | 4.20 | 1.02E−03 |
| ENSG00000159166 | LAD1 | 1.96 | 4.22 | 1.06E−03 |
| ENSG00000052126 | PLEKHA5 | 3.09 | 4.26 | 1.15E−03 |
| ENSG00000119508 | NR4A3 | 5.81 | 4.15 | 1.72E−03 |
| ENSG00000272168 | CASC15 | 1.71 | 4.52 | 1.75E−03 |
| ENSG00000154655 | L3MBTL4 | 4.17 | 4.13 | 2.57E−03 |
| ENSG00000129116 | PALLD | 8.39 | 4.09 | 2.83E−03 |
| ENSG00000135916 | ITM2C | 1.37 | 4.17 | 2.90E−03 |
| ENSG00000224228 | AL031599.1 | 1.54 | 5.32 | 4.66E−03 |
| ENSG00000166165 | CKB | 5.88 | 4.09 | 5.12E−03 |
| ENSG00000186891 | TNFRSF18 | 1.90 | 4.08 | 6.04E−03 |
| ENSG00000158457 | TSPAN33 | 5.05 | 3.86 | 6.97E−03 |
| ENSG00000013374 | NUB1 | 14.22 | 3.90 | 9.23E−03 |
| ENSG00000178175 | ZNF366 | 1.60 | 3.88 | 9.66E−03 |
| ENSG00000186827 | TNFRSF4 | 2.82 | 3.88 | 1.17E−02 |
| ENSG00000118922 | KLF12 | 1.09 | 3.91 | 1.26E−02 |
| ENSG00000273143 | AL355512.1 | 1.27 | 3.82 | 1.35E−02 |
| ENSG00000231389 | HLA-DPA1 | 24.18 | 3.83 | 1.36E−02 |
| ENSG00000172724 | CCL19 | 3.32 | 5.98 | 1.53E−02 |
| ENSG00000196562 | SULF2 | 1.30 | 3.81 | 1.60E−02 |
| ENSG00000125257 | ABCC4 | 3.19 | 3.74 | 1.70E−02 |
| ENSG00000223865 | HLA-DPB1 | 20.29 | 3.78 | 1.71E−02 |
| ENSG00000180758 | GPR157 | 2.92 | 3.68 | 1.92E−02 |
| ENSG00000151150 | ANK3 | 2.21 | 3.90 | 1.95E−02 |
| ENSG00000227591 | HSD11B1-AS1 | 1.36 | 3.76 | 2.11E−02 |
| ENSG00000234883 | MIR155HG | 5.68 | 3.68 | 2.23E−02 |
| ENSG00000164136 | IL15 | 6.85 | 3.62 | 2.25E−02 |
| ENSG00000196735 | HLA-DQA1 | 18.78 | 3.71 | 2.48E−02 |
| ENSG00000175130 | MARCKSL1 | 11.29 | 3.70 | 2.57E−02 |
| ENSG00000159231 | CBR3 | 1.78 | 3.71 | 2.58E−02 |
| ENSG00000165695 | AK8 | 2.62 | 3.60 | 2.73E−02 |
| ENSG00000179344 | HLA-DQB1 | 9.21 | 3.55 | 2.86E−02 |
| ENSG00000128487 | SPECC1 | 2.31 | 3.56 | 3.09E−02 |
| ENSG00000139146 | SINHCAF | 5.19 | 3.48 | 3.82E−02 |
| ENSG00000155962 | CLIC2 | 1.78 | 3.56 | 3.87E−02 |
| ENSG00000023445 | BIRC3 | 21.54 | 3.59 | 4.08E−02 |
| ENSG00000073803 | MAP3K13 | 7.73 | 3.44 | 4.69E−02 |
| ENSG00000154511 | DIPK1A | 1.49 | 3.50 | 4.92E−02 |

The 76 genes with higher expression in HLA-DQ2 cells were input into iPathwayGuide, which identified these genes to be involved in multiple gene ontology (GO) processes related to immune cell activation, interactions and antigen presentation (top 5 GO processes shown in Table 7).

TABLE 7
Enrichment of GO processes in genes with
increased expression in HLA-DQ2 cells.

| | GO | |
|---|---|---|
| Biological Process | Identifier | p-value |
| Positive regulation of cell-cell adhesion | GO: 0022409 | 4.0 × 10⁻¹⁰ |
| MHC class II protein complex assembly | GO: 0002399 | 2.3 × 10⁻⁹ |
| Peptide antigen assembly with MHC class | GO: 0002503 | 2.3 × 10⁻⁹ |
| II protein complex | | |
| Positive regulation of leukocyte activation | GO: 0002696 | 3.0 × 10⁻⁹ |
| Positive regulation of leukocyte activation | GO: 0050867 | 4.2 × 10⁻⁹ |

Collectively, these results indicate that HLA-DQA2 and HLA-DQB2 are expressed predominantly in dendritic cells or dendritic-like cells that also have high levels of expression of genes involved in antigen presentation.

### REFERENCES

The following references, some of which are cited above by number, as herein incorporated by reference in their entireties.
1. Network, G. A. The Global Asthma Report. (2018).
2. Demenais, F. et al. Multiancestry association study identifies new asthma risk loci that colocalize with immune-cell enhancer marks. Nat. Genet. 50, 42-53 (2018).
3. Pividori, M., Schoettler, N., Nicolae, D. L., Ober, C. & Im, H. K. Shared and distinct genetic risk factors for childhood-onset and adult-onset asthma: genome-wide and transcriptome-wide studies. Lancet Respir. Med. 7, 509-522 (2019).
4. Olafsdottir, T. A. et al. Eighty-eight variants highlight the role of T cell regulation and airway remodeling in asthma pathogenesis. Nat. Commun. 11, 393 (2020).
5. Ferreira, M. A. R. et al. Genetic Architectures of Childhood- and Adult-Onset Asthma Are Partly Distinct. Am. J. Hum. Genet. 104, 665-684 (2019).
6. Daya, M. et al. Association study in African-admixed populations across the Americas recapitulates asthma risk loci in non-African populations. Nat. Commun. 10, 1-13 (2019).
7 Bycroft, C. et al. The UK Biobank resource with deep phenotyping and genomic data. Nature 562, 203-209 (2018).
8. Schoettler, N., Rodríguez, E., Weidinger, S. & Ober, C. Advances in asthma and allergic disease genetics: Is bigger always better? J. Allergy Clin. Immunol. 144, 1495-1506 (2019).
9 Simmonds, M. & Gough, S. The HLA Region and Autoimmune Disease: Associations and Mechanisms of Action. Curr. Genomics 8, 453-465 (2009).
10. Mosaad, Y. M. Clinical Role of Human Leukocyte Antigen in Health and Disease. Scand. J. Immunol. 82, 283-306 (2015).
11. Blackwell, J. M., Jamieson, S. E. & Burgner, D. HLA and infectious diseases. Clinical Microbiology Reviews 22, 370-385 (2009).
12. Trowsdale, J. & Knight, J. C. Major Histocompatibility Complex Genomics and Human Disease. Annu. Rev. Genomics Hum. Genet. 14, 301-323 (2013).
13. Dendrou, C. A., Petersen, J., Rossjohn, J. & Fugger, L. HLA variation and disease. Nat. Rev. Immunol. 18, 325-339 (2018).
14. Wang, G., Sarkar, A., Carbonetto, P. & Stephens, M. A simple new approach to variable selection in regression, with application to genetic fine mapping. J. R. Stat. Soc. Ser. B (Statistical Methodol. 82, 1273-1300 (2020).
15. Benner, C. et al. Prospects of Fine-Mapping Trait-Associated Genomic Regions by Using Summary Statistics from Genome-wide Association Studies. Am. J. Hum. Genet. 101, 539-551 (2017).
16. Westra, H.-J. et al. Fine-mapping and functional studies highlight potential causal variants for rheumatoid arthritis and type 1 diabetes. Nat. Genet. 50, 1366-1374 (2018).
17. Mahajan, A. et al. Fine-mapping type 2 diabetes loci to single-variant resolution using high-density imputation and islet-specific epigenome maps. Nat. Genet. 50, 1505-1513 (2018).
18. Cruz-Tapias, P., Castiblanco, J. & Anaya, J.-M. HLA Association with Autoimmune Diseases. in Autoimmunity: From Bench to Bedside [Internet] (eds. Anaya, J.-M. et al.) (El Rosario University Press, 2013).
19. Jin, Y. et al. Early-onset autoimmune vitiligo associated with an enhancer variant haplotype that upregulates class II HLA expression. Nat. Commun. 10, 391 (2019).
20. Raj, P. et al. Regulatory polymorphisms modulate the expression of HLA class II molecules and promote autoimmunity. Elife 5, (2016).
21. Apps, R. et al. Influence of HLA-C expression level on HIV control. Science (80-.). 340, 87-91 (2013).
22. D'Antonio, M. et al. Systematic genetic analysis of the MHC region reveals mechanistic underpinnings of HLA type associations with disease. Elife 8, (2019).
23. Gutierrez-Arcelus, M. et al. Allele-specific expression changes dynamically during T cell activation in HLA and other autoimmune loci. Nature Genetics 52, 247-253 (2020).
24. Aguiar, V. R. C., César, J., Delaneau, O., Dermitzakis, E. T. & Meyer, D. Expression estimation and eQTL mapping for HLA genes with a personalized pipeline. PLOS Genet. 15, e1008091 (2019).
25. Dunham, I. et al. An integrated encyclopedia of DNA elements in the human genome. Nature 489, 57-74 (2012).
26. MacArthur, J. et al. The new NHGRI-EBI Catalog of published genome-wide association studies (GWAS Catalog). Nucleic Acids Res. 45, D896-D901 (2017).
27. Moffatt, M. F. et al. A large-scale, consortium-based genomewide association study of asthma. N. Engl. J. Med. 363, 1211-1221 (2010).
28. Li, X. et al. Genome-wide association study of asthma identifies RAD50-IL13 and HLA-DR/DQ regions. J. Allergy Clin. Immunol. 125, 328-335.e11 (2010).
29. Lasky-Su, J. et al. HLA-DQ strikes again: Genome-wide association study further confirms HLA-DQ in the diagnosis of asthma among adults. Clin. Exp. Allergy 42, 1724-1733 (2012).
30. Aguet, F. et al. Genetic effects on gene expression across human tissues. Nature 550, 204-213 (2017).
31. Waage, J. et al. Genome-wide association and HLA fine-mapping studies identify risk loci and genetic pathways underlying allergic rhinitis. Nat. Genet. 50, 1072-1080 (2018).
32. Calderon, D. et al. Landscape of stimulation-responsive chromatin across diverse human immune cells. Nat. Genet. 51, 1494-1505 (2019).
33. Blais, M. E., Dong, T. & Rowland-Jones, S. HLA-C as a mediator of natural killer and T-cell activation: Spectator or key player? Immunology 133, 1-7 (2011).
34. Fadda, L. et al. Peptide antagonism as a mechanism for NK cell activation. Proc. Natl. Acad. Sci. U.S.A 107, 10160-10165 (2010).
35. Karimi, K. & Forsythe, P. Natural killer cells in asthma. Frontiers in Immunology 4, 159 (2013).
36. Kim, J. H. & Jang, Y. J. Role of natural killer cells in airway inflammation. Allergy, Asthma and Immunology Research 10, 448-456 (2018).
37. Li, X. et al. Genome-wide association studies of asthma indicate opposite immunopathogenesis direction from autoimmune diseases. J. Allergy Clin. Immunol. 130, 861-8.e7 (2012).
38. Berdoz, J., Tiercy, J.-M., Rollini, P., Mach, B. & Gorski, J. Remarkable sequence conservation of the HLA-DQB2 locus (DX beta) within the highly polymorphicDQ subregion of the human MHC. Immunogenetics 29, 241-248 (1989).
39. Gaur, L. K., Heise, E. R., Thurtle, P. S. & Nepom, G. T. Conservation of the HLA-DQB2 locus in nonhuman primates. J. Immunol. 148, 943-8 (1992).
40. Lenormand, C. et al. HLA-DQA2 and HLA-DQB2 Genes Are Specifically Expressed in Human Langerhans Cells and Encode a New HLA Class II Molecule. J. Immunol. 188, 3903-3911 (2012).
41. Gamazon, E. R. et al. A gene-based association method for mapping traits using reference transcriptome data. Nat. Genet. 47, 1091-1098 (2015).
42. Mine, K. L. et al. Heightened expression of HLA-DQB1 and HLA-DQB2 in pre-implantation biopsies predicts poor late kidney graft function. Hum. Immunol. 79, 594-601 (2018).
43. Farina et al. HLA-DQA1 and HLA-DQB1 Alleles, Conferring Susceptibility to Celiac Disease and Type 1 Diabetes, are More Expressed Than Non-Predisposing Alleles and are Coordinately Regulated. Cells 8, 751 (2019).
44. Pirinen, M., Donnelly, P. & Spencer, C. C. A. Efficient computation with a linear mixed model on large-scale data sets with applications to genetic studies. Ann. Appl. Stat. 7, 369-390 (2013).
45. Benner, C. et al. FINEMAP: efficient variable selection using summary data from genome-wide association studies. Bioinformatics 32, 1493-1501 (2016).
46. Banerjee, S., Zeng, L., Schunkert, H. & Söding, J. Bayesian multiple logistic regression for case-control GWAS. PLOS Genet. 14, e1007856 (2018).
47. Gourraud, P.-A. et al. HLA Diversity in the 1000 Genomes Dataset. PLOS One 9, e97282 (2014).
48. Luo, Y. et al. A high-resolution HLA reference panel capturing global population diversity enables multi-ethnic fine-mapping in HIV host response. medRxiv (2020). doi: 10.1101/2020.07.16.20155606
49. Trivedi, M. & Denton, E. Asthma in children and adults-what are the differences and what can they tell us about asthma? Frontiers in Pediatrics 7, 256 (2019).
50. Jia, X. et al. Imputing Amino Acid Polymorphisms in Human Leukocyte Antigens. PLOS One 8, e64683 (2013).
51. Cusanovich, D. A. et al. Integrated analyses of gene expression and genetic association studies in a founder population. Hum. Mol. Genet. 25, 2104-2112 (2016).
52. Weitkamp, L. & Ober, C. Ancestral and recombinant 16-locus HLA haplotypes in the Hutterites. Immunogenetics 49, 491-7 (1999).
53. Robinson, J. et al. The IPD and IMGT/HLA database: allele variant databases. Nucleic Acids Res. 43, D423-31 (2015).
54. van de Geijn, B., McVicker, G., Gilad, Y. & Pritchard, J. K. WASP: allele-specific software for robust molecular quantitative trait locus discovery. Nat. Methods 12, 1061-3 (2015).
55. Dobin, A. et al. STAR: ultrafast universal RNA-seq aligner. Bioinformatics 29, 15-21 (2013).
56. Law, C. W., Chen, Y., Shi, W. & Smyth, G. K. voom: precision weights unlock linear model analysis tools for RNA-seq read counts. Genome Biol. 15, R29 (2014).
57. Ritchie, M. E. et al. limma powers differential expression analyses for RNA-sequencing and microarray studies. Nucleic Acids Res. 43, e47-e47 (2015).
58. Zhou, X. & Stephens, M. Genome-wide efficient mixed-model analysis for association studies. Nat. Genet. 44, 821-4 (2012).
59. Gern, J. E. et al. The Urban Environment and Childhood Asthma (URECA) birth cohort study: design, methods, and study population. BMC Pulm. Med. 9, 17 (2009).
60. Altman, M. C. et al. Allergen-induced activation of natural killer cells represents an early-life immune response in the development of allergic asthma. J. Allergy Clin. Immunol. 142, 1856-1866 (2018).
61. Dilthey, A. T. et al. HLA*LA-HLA typing from linearly projected graph alignments. Bioinformatics 35, 4394-4396 (2019).
62. Delaneau, O. et al. A complete tool set for molecular QTL discovery and analysis. Nat. Commun. 8, 1-7 (2017).
63. Altman, M. et al. Airway Epithelium Gene Expression Endotyping of Asthma and Airway Obstruction in Urban Children [abstract]. J. Allergy Clin. Immunol. 145, AB176 (2020).
64. Magnaye, K. M. et al. A-to-I editing of miR-200b-3p in airway cells is associated with moderate-to-severe asthma. Eur. Respir. J. (in Press. (2021).
65. Rich, S. S. et al. The type 1 diabetes genetics consortium. Ann. N. Y. Acad. Sci. 1079, 1-8 (2006).
66. Berman, H. M. et al. The Protein Data Bank. Nucleic Acids Res. 28, 235-242 (2000).
67. Kaur, G. et al. Structural and regulatory diversity shape HLA-C protein expression levels. Nat. Commun. 8, (2017).
68. Jiang, W. et al. In vivo clonal expansion and phenotypes of hypocretin-specific CD4+ T cells in narcolepsy patients and controls. Nat. Commun. 10, 1-17 (2019).
69. Tollefsen, S. et al. Structural and functional studies of trans-encoded HLA-DQ2.3 (DQA1*03:01/DQB1*02:01) protein molecule. J. Biol. Chem. 287, 13611-13619 (2012).

1. A method of treating or preventing adult-onset asthma (AOA) or another disease or condition comprising inhibiting the activity and/or the expression of human leukocyte antigen (HLA) class II histocompatibility antigen, DQ alpha 2 chain (HLA-DQA2) and/or HLA class II histocompatibility antigen, DQ beta 2 chain (HLA-DQB2).

2. The method of claim 1, wherein the activity and/or the expression of HLA-DQA2 and/or HLA-DQB2 is inhibited in lung epithelial cells and/or Langerhans cells.

3. The method of claim 1, wherein inhibiting the activity of HLA-DQA2 and/or HLA-DQB2 comprises inhibiting the binding of one or more antigenic peptides present in HLA-DQA2 and/or HLA-DQB2 from binding and/or being recognized by immune cells.

4. The method of claim 3, wherein inhibiting the activity of HLA-DQA2 and/or HLA-DQB2 comprises administering a HLA-DQA2 and/or HLA-DQB2 inhibitor.

5. The method of claim 4, wherein the HLA-DQA2 and/or HLA-DQB2 inhibitor is a small molecule, peptide, or antibody.

6. A composition comprising an HLA-DQA2 and/or HLA-DQB2 inhibitor for use in the method of one of claims 1-5.

7. The method of claim 1, wherein inhibiting the expression of HLA-DQA2 and/or HLA-DQB2 comprises targeting the HLA-DQA2 and/or HLA-DQB2 genes and reducing expression thereof.

8. The method of claim 7, wherein reducing expression of HLA-DQA2 and/or HLA-DQB2 comprises administering a nucleic acid inhibitor of gene expression.

9. A composition comprising the nucleic acid inhibitor of HLA-DQA2 and/or HLA-DQB2 gene expression of claim 8.

10. The method of claim 7, wherein targeting the HLA-DQA2 and/or HLA-DQB2 genes comprises knocking down expression by RNAi or CRISPR.

11. The method of claim 1, wherein inhibiting the expression of HLA-DQA2 and/or HLA-DQB2 comprises targeting one or more single nucleotide polymorphisms (SNPs) that are causal variants of the increased expression of HLA-DQA2 and/or HLA-DQB2.

12. The method of claim 11, wherein the one or more SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and rs9274660.

13. The method of claim 12, wherein targeting the one or more SNPs comprises editing one or more of the genes containing the SNPs.

14. The method of claim 13, wherein one or more of the genes containing the SNPs is edited by CRISPR.

15. A composition comprising gene editing agents for use in the method of one of claims 13-14.

16. The method of claim 1, wherein the other disease or condition is selected from an autoimmune disease, an allergy, or a cancer.

17. A method comprising:
(a) obtaining a sample from a subject;
(b) detecting the level of HLA-DQA2 and/or HLA-DQB2 gene expression in the sample; and
(c) comparing the level of HLA-DQA2 and/or HLA-DQB2 gene expression in the sample to a control or threshold level.

18. The method of claim 1617 further comprising assessing the risk of the subject developing adult-onset asthma (AOA) based on the comparison of step (c), wherein if the level of HLA-DQA2 and/or HLA-DQB2 gene expression in the sample is greater than a control or threshold level then the subject is at increased risk of developing AOA.

19. The method of claim 17, further comprising characterizing the type of asthma the subject suffers from based on the comparison of step (c), wherein if the level of HLA-DQA2 and/or HLA-DQB2 gene expression in the sample is greater than a control or threshold level then the subject suffers from a HLA-DQA2- and/or HLA-DQB2-dependent AOA.

20. The method of claim 17, further comprising determining a treatment for the subject based on the comparison of step (c), wherein an increased level of HLA-DQA2 and/or HLA-DQB2 gene expression in the sample compared to the control or threshold level indicates treatment by the compositions or methods of one of claims 1-16.

21. The method of claim 17, wherein the sample comprises lung epithelial cells and/or Langerhans cells.

22. The method of claim 17, wherein the level of HLA-DQA2 and/or HLA-DQB2 gene expression is measured by quantitative PCR (qPCR).

23. A method comprising:
(a) obtaining a sample from a subject; and
(b) detecting in the sample the presence/absence of one or more single nucleotide polymorphisms (SNPs) that are causal variants of the increased expression of HLA-DQA2 and/or HLA-DQB2.

24. The method of claim 23, further comprising assessing the risk of the subject developing adult-onset asthma (AOA) based on the presence/absence of one or more SNPs that are causal variants of the increased expression of HLA-DQA2 and/or HLA-DQB2, wherein the presence of one or more of the causal variants in the sample is indicative of an increased risk of developing AOA.

25. The method of claim 23, further comprising characterizing the type of asthma the subject suffers from based on the presence/absence of one or more SNPs that are causal variants of the increased expression of HLA-DQA2 and/or HLA-DQB2, wherein the presence of one or more of the causal variants in the sample indicates that the subject suffers from a HLA-DQA2- and/or HLA-DQB2-dependent AOA.

26. The method of claim 23, further comprising determining a treatment for the subject based on the presence/absence of one or more SNPs that are causal variants of the increased expression of HLA-DQA2 and/or HLA-DQB2, wherein the presence of one or more of the causal variants in the sample indicates treatment by the compositions or methods of one of claims 1-16.

27. The method of claim 23, wherein the sample comprises lung epithelial cells and/or Langerhans cells.

28. The method of claim 23, wherein the one or more SNPs are selected from rs9272346, rs34843907, rs9272346, rs34843907, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330 rs3828789, rs9274660, rs9273339, rs17612858, rs9272346, rs9273084, rs17843573, rs17612576, rs17843577, rs17843580, rs17612625, rs17612633, rs17612781, rs17612788, rs17612802, rs9273326, rs9273329, rs9273330, rs3828789, rs9274660, rs1063355, rs9273084, rs17843573, rs17612576, rs17843577, rs17843580, rs17612625, rs17612633, rs17612781, rs17612788, rs17612802, rs17612858, rs9273326, rs9273329, rs9273330, rs9273339, rs3828789, and rs9274660.
